(19) United States
(12) Patent Application Publication
(10) Pub. No.: US 2014/0242707 A1
(43) Pub. Date: Aug. 28, 2014

(54) Compositions and Methods for Diagnosing Hypercoagulability and Hypocoagulability

(71) Applicants: Vance G. Nielsen, Tucson, AZ (US); Keith Vosseller, Philadelphia, PA (US)

(72) Inventors: Vance G. Nielsen, Tucson, AZ (US); Keith Vosseller, Philadelphia, PA (US)

(73) Assignee: Philadelphia Health & Education Corporation d/b/a Drexel University College of Medicine, Philadelphia, PA (US)

(21) Appl. No.: 14/233,690

(22) PCT Filed: Jul. 19, 2012
(86) PCT No.: PCT/US2012/047407

§ 371 Date: Mar. 25, 2014

Related U.S. Application Data
(60) Provisional application No. 61/510,292, filed on Jul. 21, 2011.

(51) Int. Cl. G01N 33/86

(52) U.S. Cl. CPC G01N 33/86
USPC 436/69

(57) ABSTRACT

The present invention provides a method for diagnosing hypercoagulability associated with carbon monoxide exposure in a subject. The method comprises obtaining a plasma sample from the subject, dividing the sample into at least two portions, and determining the clot strength of the first portion. The method further comprises exposing the second portion to an organic reductant, determining the clot strength of the second portion after exposure to the organic reductant, and comparing the clot strength of the first portion with the clot strength of the second portion. A diagnosis of hypercogulability associated with carbon monoxide exposure is made when the clot strength of the second portion is decreased compared with the cloth strength of the first portion.

### CROSS-REFERENCE TO RELATED APPLICATIONS

This application claims priority to U.S. Patent Application Ser. No. 61/510,292, filed Jul. 21, 2011, the contents of which are incorporated by reference herein in its entirety.

### BACKGROUND OF THE INVENTION

Cigarette smoke is associated with plasmatic hypercoagulability, and remains a serious cause of morbidity and mortality worldwide. In particular, the inflammatory and prothrombotic state associated with smoking is strongly linked with myocardial infarction and stroke (Lind et al., 2004, Arterioscler Thromb Vasc Biol 24:577-582). The smoking-induced systemic hypercoagulable state involves an increase in circulating fibrinogen (Lind et al., 2004, Arterioscler Thromb Vasc Biol 24:577-582), an increase in circulating factor XIII (FXIII) (Ariens et al., 1999, Arterioscler Thromb Vasc Biol 19:2012-2016; van Wersch et al., 1997, Int J Clin Lab Res 27:68-71), and enhanced activation of circulating platelets (Neubauer et al., 2009, Blood Coagul Fibrinolysis 20:694-698). Further, the microarchitecture of clots formed from smoker's blood demonstrated a denser matrix of thin fibrin polymers and greater clot strength via thrombelastography compared to nonsmokers (Barua et al., 2010, Arterioscler Thromb Vasc Biol 30:75-79). One likely mechanism involved in smoking-associated thrombophilia is exposure of plasma proteins to carbon monoxide (CO), which has been documented to coexist in these hypercoagulable states either as carboxyhemoglobin or exhaled carbon monoxide gas (Lind et al., 2004, Arterioscler Thromb Vasc Biol 24: 577-582). Further, CO has been demonstrated to enhance coagulation by binding to a fibrinogen-bound heme.

Conversely, hypocoagulation can be caused by exposure to nitric oxide (NO), which can result in reduced clot strength and inability to stop bleeding effectively. NO-mediated hypocoagulation can occur in a wide variety of clinical settings, including those on chronic nitrates, those with systemic inflammation, and those needing NO inhalation.

Given the potential for extreme adverse events for those with either hypercoagulable or hypocoagulable conditions, and the lack of current options for detecting a subject's coagulation state, there is a need in the art for methods of detecting those at risk for having or developing hypercoagulation or hypocogulation. The present invention addresses this unmet need in the art.

### BRIEF SUMMARY OF THE INVENTION

The present invention provides a method for diagnosing hypercoagulability associated with carbon monoxide exposure in a subject. The method comprises obtaining a plasma sample from the subject, dividing the sample into at least two portions, and determining the clot strength of the first portion. The method further comprises exposing the second portion to an organic reductant, determining the clot strength of the second portion after exposure to the organic reductant, and comparing the clot strength of the first portion with the clot strength of the second portion. A diagnosis of hypercoagulability associated with carbon monoxide exposure is made when the clot strength of the second portion is decreased compared with the clot strength of the first portion.

In one embodiment, the hypercoagulability is associated with the formation of carboxyhemefibrinogen.

In one embodiment, the clot strength of the first portion is greater than an institutionally generated normal 95% confidence interval. In one embodiment, the clot strength of the second portion is decreased by at least 35% compared to the first portion.

In some embodiments, the strength of the clot is determined by measuring the elastic modulus (G). In some embodiments, the clot strength is determined by using a thromboelastograph or a thromboelastometer. In some embodiments, the subject is a mammal.

In one embodiment, the organic reductant is one of phenyhydroxylamine, phenylhydrazine, sym-diphenylhydrazine, 1,4-cyclohexane, hydroquinone, benzhydrol, methylhydrazine, 2-methyl-1,3-cyclopentanedione, acetylacetone, isopropyl alcohol, benzaldehyde, or malononitrile. In some embodiments, the amount of organic reductant is sufficient to produce a concentration of 30 mM. In another embodiment, the amount of the organic reductant is sufficient to produce a concentration of 5-500 mM, 10-400 mM, 15-300 mM, 20-200 mM, or 25-100 mM.

In one embodiment, the method comprises the addition of a carbon monoxide-producing molecule to a third portion of the plasma sample. In some embodiments, when the addition of the carbon monoxide-producing molecule to the third portion does not increase the clot strength of the third portion by at least 35% compared to the clot strength of the first sample, the diagnosis of hypercoagulability associated with carbon monoxide exposure is confirmed.

In some embodiments, the carbon monoxide producing molecule is one of tricarbonyldichlororuthenium (II) dimer ([RuCl2(CO)3]2; carbon monoxide releasing molecule-2; CORM-2), iron pentacarbonyl (Fe(CO)5), dimanganese decacarbonyl (Mn2(CO)10; CORM-1), tricarbonylchloro(glycinato)ruthenium (II) (Ru(CO)3Cl(glycinate); CORM-3), or sodium boranocarbonate (Na2[H3BCO2]; CORM-A1). In some embodiments, the amount of the carbon monoxide producing molecule is sufficient to produce a concentration of 100 μM. In another embodiment, the amount of the carbon monoxide producing molecule is sufficient to produce a concentration of 5-500 μM, 10-400 μM, 15-300 μM, 20-200 μM, or 25-100 μM.

In another aspect, the present invention provides a method for diagnosing hypocoagulability associated with nitric oxide exposure in a subject. The method comprises obtaining a plasma sample from the subject, dividing the sample into at least two portions, and determining the clot strength of the first portion. The method further comprises exposing the second portion to a carbon monoxide producing molecule, determining the clot strength of the second portion after exposure to the carbon monoxide producing molecule, and comparing the clot strength of the first portion with the clot strength of the second portion. A diagnosis of hypocoagulability associated with nitric oxide exposure is made when the clot strength of the second portion is increased compared with the clot strength of the first portion.

In one embodiment, the hypocoagulability is associated with the formation of methemefibrinogen.

In one embodiment, the clot strength of the first portion is less than an institutionally generated normal 95% confidence interval. In one embodiment, the clot strength of the second portion is increased by at least 160% compared to the first portion.

In some embodiments, the strength of the clot is determined by measuring the elastic modulus (G). In some embodiments, the clot strength is determined by using a thromboelastograph or a thromboelastometer. In some embodiments, the subject is a mammal.

In some embodiments, the carbon monoxide producing molecule is one of tricarbonyldichlororuthenium (II) dimer ([RuCl2(CO)3]2; carbon monoxide releasing molecule-2; CORM-2), iron pentacarbonyl (Fe(CO)5), dimanganese decacarbonyl (Mn2(CO)10; CORM-1), tricarbonylchloro(glycinato)ruthenium (II) (Ru(CO)3Cl(glycinate); CORM-3), or sodium boranocarbonate (Na2[H3BCO2]; CORM-A1). In some embodiments, the amount of the carbon monoxide producing molecule is sufficient to produce a concentration of 100 μM. In another embodiment, the amount of the carbon monoxide producing molecule is sufficient to produce a concentration of 5-500 μM, 10-400 μM, 15-300 μM, 20-200 μM, or 25-100 μM.

In one embodiment, the method comprises the addition of an organic reductant to a third portion of the plasma sample. In some embodiments, when the addition of the organic reductant to the third portion does not decrease the clot strength of the third portion by at least 35% compared to the clot strength of the first sample, the diagnosis of hypocoagulability associated with nitric oxide exposure is confirmed.

In one embodiment, the organic reductant is one of phenyhydroxylamine, phenylhydrazine, sym-diphenylhydrazine, 1,4-cyclohexane, hydroquinone, benzhydrol, methylhydrazine, 2-methyl-1,3-cyclopentanedione, acetylacetone, isopropyl alcohol, benzaldehyde, or malononitrile. In some embodiments, the amount of organic reductant is sufficient to produce a concentration of 30 mM. In another embodiment, the amount of the organic reductant is sufficient to produce a concentration of 5-500 mM, 10-400 mM, 15-300 mM, 20-200 mM, or 25-100 mM.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts the results of experiments demonstrating an increase in the elastic modulus (G, dynes/cm²) (a measure of clot strength) of clots formed in the presence of 100 μM CORM-2, and further depicts reduction of G in blood pretreated with 10 mM phenyhydroxylamine (PHA) for 2 minutes, in both control conditions as well as when CORM-2 was added concurrently.

FIG. 2 depicts the results of experiments demonstrating an increase in the elastic modulus (G, dynes/cm²) (a measure of clot strength) of clots formed in the presence of 100 μM CORM-2, and further depicts reduction of G in blood treated with 30 mM PHA 5 minutes after CORM-2 addition.

FIG. 3 depicts the results of experiments demonstrating that pretreatment with 30 mM PHA prevented CORM-2 mediated increases in G.

FIG. 4 depicts the results of experiments demonstrating the increase in G of clots formed in the presence of increasing concentrations of CORM-2 and in the absence of PHA.

FIG. 5 depicts the results of experiments demonstrating the values for time to maximum rate of thrombus formation (TMRTG), maximum rate of thrombus generation (MRTG) and clot strength, as given by total thrombus generation (TTG) for normal and smoker plasma.

FIG. 6 depicts the results of experiments demonstrating the changes in clot strength after plasma exposure to CORM-2 and PHA, for normal and smoker plasma.

FIG. 7, comprising FIG. 7A and FIG. 7B, depicts the results of experiments demonstrating thrombus formation velocity curves. FIG. 7A depicts results from a sample of normal, pooled plasma. FIG. 7B displays the results obtained from plasma obtained from a patient with hemolysis and thrombosis of a Heartmate II ventricular assist device. MRTG=maximum rate of thrombus formation, a measure of velocity of clot formation; CORM=addition of 100 μM CORM-2; Control=addition of dH₂O; PHA=addition of 30 mM phenylhydroxylamine.

FIG. 8 depicts the results of experiments demonstrating the comparison of changes in clot strength in normal and patient plasma. TTG=total thrombus generation, a measure of clot strength; normal=normal pooled plasma; HMII=plasma results from patient with thrombosed Heartmate II ventricular assist device. COHb=carboxyhemoglobin concentration; MetHb=methemoglobin concentration; CORM=addition of 100 μM CORM-2; Control=addition of dH₂O; PHA=addition of 30 mM phenylhydroxylamine.

### DETAILED DESCRIPTION

The invention provides methods of diagnosing hypercoagulability associated with exposure to carbon monoxide and hypocoagulability associated with exposure to nitric oxide. In various embodiments, the method comprises diagnostic assays to detect carboxyhemefibrinogen and methemefibrinogen by modulating the redox state of fibrinogen-bound heme.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, time of day, etc.) from those organisms, tissues, cells or components thereof that display the “normal” (expected) respective characteristic. Characteristics which are normal or expected for one organism, cell or tissue type, might be abnormal for a different organism, cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The terms “patient,” “subject,” “individual,” and the like are used interchangeably herein, and refer to any animal, amenable to the methods described herein. In certain non-limiting embodiments, the patient, subject or individual is a human.

As used herein, an “instructional material” includes a publication, a recording, a diagram, or any other medium of expression which can be used to communicate the usefulness of a compound, composition, or method of the invention in the kit for effecting alleviation of the various diseases or disorders recited herein. The instructional material of the kit of the invention can, for example, be affixed to a container which contains the identified compound or composition of the invention or be shipped together with a container which contains the identified compound or composition. Alternatively, the instructional material can be shipped separately from the container with the intention that the instructional material and the compound be used cooperatively by the recipient.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The invention includes methods of diagnosing hypercoagulability associated with exposure of a subject to carbon monoxide (CO). Exposure to CO can occur through exogenous exposure, for example through the presence of CO in the local environment. Further, exposure to CO can occur through endogenous production, for example through the body's own production of CO during various clinical states. The present invention can detect CO-mediated hypercoagulability that may occur during either exogenous or endogenous exposure. In one embodiment, the invention provides methods of assessing the risk for hypercoagulability in a subject. In another embodiment, the invention provides methods of monitoring hypercoagulability in a subject.

The invention also provides methods of diagnosing hypocoagulability associated with exposure of a subject to nitric oxide (NO). Exposure to NO can occur through exogenous exposure, for example through the presence of NO in the local environment or through therapeutic administration of NO and other nitrates. Further, exposure to NO can occur through endogenous production, for example through the body's own production of NO during various clinical states. The present invention can detect NO-mediated hypocoagulability that may occur during either exogenous or endogenous exposure. In one embodiment, the invention provides methods of assessing the risk for hypocoagulability in a subject. In another embodiment, the invention provides methods of monitoring hypocoagulability in a subject.

### Methods of Diagnosing Hypercoagulability

In one embodiment, the invention provides a method of diagnosing hypercoagulability associated with carbon monoxide exposure in a subject. In various embodiments, the method comprises diagnostic assays to detect carboxyhemefibrinogen by modulating the redox state of fibrinogen-bound heme. For the methods of the invention described herein, plasma is obtained from a subject by collection of a blood sample, which is subjected to centrifugation to isolate the plasma. However, the invention is not limited to a particular method of obtaining plasma. Rather, plasma may be obtained by way of any method known in the art. Exemplary methods include plasmapheresis, discontinuous flow centrifugation, continuous flow centrifugation, plasma filtration, or combinations thereof. The methods of diagnosing described herein, can also be used to monitor, over time, a subject who is recovering from hypercoagulability, as well as to evaluate the effectiveness of an applied treatment of hypercoagulability.

In one embodiment, the invention comprises determining the clot strength of a plasma sample obtained from a subject, and comparing it with the clot strength of a comparator sample. In some embodiments, the clot strength of the subject's plasma sample, and the comparator sample, is determined by measuring the elastic modulus (G) of the sample. In various embodiments, the comparator sample is at least one of a positive control, a negative control, a standard having a known clot strength value, a plasma sample with an organic reductant added, a plasma sample with a carbon-monoxide producing molecule added, a historical norm or an institutionally generated normal 95% confidence interval. In some embodiments, clot strength is determined using a thromboelastometer or a thromblastograph.

In some embodiments, the invention comprises exposing plasma to an effective amount of an organic reductant and determining its clot strength, before and/or after its exposure to the organic reductant. In one embodiment, the organic reductant is phenyhydroxylamine (PHA). In various embodiments, the organic reductant is PHA, phenylhydrazine, sym-diphenylhydrazine, 1,4-cyclohexane, hydroquinone, benzhydrol, methylhydrazine, 2-methyl-1,3-cyclopentanedione, acetylacetone, isopropyl alcohol, benzaldehyde, malononitrile or a combination thereof.

In a one embodiment, the effective amount of the organic reductant, PHA, is sufficient to produce a concentration of 30 mM. In various embodiments, the effective amount of the reductant is sufficient to produce a concentration of 5-500 mM, 10-400 mM, 15-300 mM, 20-200 mM, or 25-100 mM.

In a further embodiment, the invention comprises exposing plasma to an effective amount of a carbon monoxide-producing molecule or a carbon monoxide donor. In one embodiment, the carbon monoxide-producing molecule is tricarbonyldichlororuthenium (II) dimer ([RuCl2(CO)3]2; carbon monoxide releasing molecule-2; CORM-2). In various embodiments, the carbon monoxide-producing molecule is tricarbonyldichlororuthenium (II) dimer ([RuCl2(CO)3]2; carbon monoxide releasing molecule-2; CORM-2), iron pentacarbonyl (Fe(CO)₅), dimanganese decacarbonyl (Mn2(CO)10; CORM-1), tricarbonylchloro(glycinato)ruthenium (II) (Ru(CO)3Cl(glycinate); CORM-3), sodium boranocarbonate (Na2[H3BCO2]; CORM-A1), or a combination thereof.

In one embodiment, the effective amount of the carbon monoxide-producing molecule, CORM-2, is sufficient to produce a concentration of 100 μM. In various embodiments, the effective amount of the carbon monoxide-producing molecule is sufficient to produce a concentration of 5-600 μM, 10-500 μM, 15-400 μM, 20-300 μM, 25-200 μM or 30-100 μM.

In one embodiment, diagnosing hypercoagulability comprises detecting that the clot strength of the subject plasma is greater than the clot strength of plasma obtained from a subject known to not have hypercoagulability. In one embodiment, diagnosing hypercoagulability comprises detecting that clot strength of the subject plasma is greater than an institutionally generated 95% confidence interval.

In one embodiment, diagnosing hypercoagulability comprises detecting that the clot strength of the subject plasma substantially decreases upon exposure to an organic reductant (e.g. PHA). In one embodiment, hypercoagulability is determined by evaluating the PHA-mediated decrease in the clot strength of a sample, wherein the PHA-mediated decrease is given by the percent decrease in clot strength of PHA treated plasma compared to untreated plasma obtained from the same source. In one embodiment, hypercoagulability is diagnosed if the PHA treated plasma exhibits a clot strength that is decreased by at least 30% compared to the subject's untreated plasma. In another embodiment, hypercoagulability is diagnosed if the PHA treated plasma exhibits a clot strength that is decreased by at least 35% compared to the subject's untreated plasma. In another embodiment, hypercoagulability is diagnosed if the PHA treated plasma exhibits a clot strength that is decreased by at least 40% compared to the subject's untreated plasma. In another embodiment, hypercoagulability is diagnosed if the PHA treated plasma exhibits a clot strength that is decreased by at least 45% compared to the subject's untreated plasma.

In one embodiment, diagnosing hypercoagulability comprises detecting that the clot strength of the subject plasma does not substantially increase upon exposure to carbon monoxide producing molecule (e.g. CORM-2). In one embodiment, hypercoagulability is determined by evaluating the CORM-2-mediated increase in the clot strength of a sample, wherein the CORM-2-mediated increase is given by the percent increase in clot strength of CORM-2 treated plasma compared to untreated plasma obtained from the same source. In one embodiment, hypercoagulability is diagnosed if the CORM-2 treated plasma exhibits a clot strength that is not increased by at least 70% compared to the subject's untreated plasma. In another embodiment, hypercoagulability is diagnosed if the CORM-2 treated plasma exhibits a clot strength that is not increased by at least 60% compared to the subject's untreated plasma. In another embodiment, hypercoagulability is diagnosed if the CORM-2 treated plasma exhibits a clot strength that is not increased by at least 50% compared to the subject's untreated plasma. In another embodiment, hypercoagulability is diagnosed if the CORM-2 treated plasma exhibits a clot strength that is not increased by at least 40% compared to the subject's untreated plasma. In another embodiment, hypercoagulability is diagnosed if the CORM-2 treated plasma exhibits a clot strength that is not increased by at least 30% compared to the subject's untreated plasma. In one embodiment, hypercoagulability is diagnosed if the CORM-2-mediated increase in clot strength of a subject's plasma is less than the CORM-2-mediated increase in clot strength of control plasma, obtained from a source known to not have hypercoagulability. In another embodiment, hypercoagulability is diagnosed if the CORM-2-mediated increase in clot strength of a subject's plasma is less than an institutionally generated 95% confidence interval of CORM-2-mediated increases in clot strength.

A subject in need of assessment of the risk of developing hypercoagulability associated with carbon monoxide exposure by the methods described herein includes any subject exposed or potentially exposed, acutely or chronically, to carbon monoxide.

A subject in need of assessment of the risk of developing hypercoagulability includes those exposed or potentially exposed to exogenous carbon monoxide. For example, subjects may be exposed to higher than normal carbon monoxide exogenously present in their local environments. By way of non-limiting examples a subject in need of assessment of the risk of developing hypercoagulability associated with exposure to carbon monoxide include, but are not limited to, smokers, second-hand smokers, bartenders, waitresses, casino workers, family members of smokers, firefighters, residents living in poor air quality locations, welders, garage mechanics, carbon-black makers, miners, organic chemical synthesizers, metal oxide reducers, longshore workers, diesel engine operators, forklift operators, marine terminal workers, toll-both attendants, tunnel attendants, customs inspectors, police officers, taxi drivers, factory workers, boiler room workers, brewery workers, those working in paper production, those working in steel production, those working on or around docks, those working in or around grain elevators and silos, those working near blast furnaces, and those working near coke ovens.

Further, a subject in need of assessment of the risk of developing hypercoagulability includes those exposed or potentially exposed to endogenously produced carbon monoxide. There are several disease states that involve consequent exposure to carbon monoxide secondary to upregulation of the hemoglobin catabolic pathway. One enzyme in particular, hemoxygenase-1 (HO-1) generates carbon monoxide when breaking down heme released from hemoglobin. A variety of stimuli increases the activity of this enzyme, resulting in increases in observed exhaled carbonmonoxide and marked increases in circulating carboxyhemoglobin, a primary marker of carbon monoxide exposure. Disease states that increase carbon monoxide production include destructive anemia (e.g. sickle cell anemia, thalassemia, hemolytic anemia) and cancer (e.g., pancreatic cancer, glioblastoma multiforme). Other chronic systemic diseases associated with hypercoagulability, and which have been noted to involve increased carbon monoxide production or HO-1 upregulation include coronary artery disease, type 2 diabetes, uremia, and hepatic cirrhosis. Trauma/critical care patients also demonstrate increased carbon monoxide release (from ischemia-reperfusion, shock, heme release from resolving hematomas). Patients with heart failure, chronic lung disease and congestive heart failure also demonstrate increased carbon monoxide production. Furthermore, ventricular assist devices (VADs) are often associated with hypercoagulability. Therefore, non-limiting examples of a subject in need of assessment of the risk of developing hypercoagulability associated with endogenous carbon monoxide production includes trauma patients, critical care patients, patients who have received a VAD, as well as those having or suspected to have anemia, cancer, coronary artery disease, type 2 diabetes, uremia, hepatic cirrhosis, heart failure, chronic lung disease and congestive heart failure.

### Methods of Diagnosing Hypocoagulability

In one embodiment, the invention provides a method of diagnosing hypocoagulability associated with nitric oxide exposure in a subject. In various embodiments, the method comprises diagnostic assays to detect methemefibrinogen by modulating the redox state of fibrinogen-bound heme. For the methods of the invention described herein, plasma is obtained from a subject by collection of a blood sample, which is subjected to centrifugation to isolate the plasma. However, the invention is not limited to a particular method of obtaining plasma. Rather, plasma may be obtained by way of any method known in the art. Exemplary methods include plasmapheresis, discontinuous flow centrifugation, continuous flow centrifugation, plasma filtration, or combinations thereof. The methods of diagnosing described herein, can also be used to monitor, over time, a subject who is recovering from hypocoagulability, as well as to evaluate the effectiveness of an applied treatment of hypocoagulability.

In one embodiment, the invention comprises determining the clot strength of a plasma sample obtained from a subject, and comparing it with the clot strength of a comparator sample. In some embodiments, the clot strength of the subject's plasma sample, and the comparator sample, is determined by measuring the elastic modulus (G) of the sample. In various embodiments, the comparator sample is at least one of a positive control, a negative control, a standard having a known clot strength value, a plasma sample with an organic reductant added, a plasma sample with a carbon-monoxide producing molecule added, a historical norm or an institutionally generated normal 95% confidence interval. In some embodiments, clot strength is determined using a thromboelastometer or a thromblastograph.

In some embodiments, the invention comprises exposing plasma to an effective amount of an organic reductant and determining its clot strength, before and/or after its exposure to the organic reductant. In one embodiment, the organic reductant is phenyhydroxylamine (PHA). In various embodiments, the organic reductant is PHA, phenylhydrazine, sym-diphenylhydrazine, 1,4-cyclohexane, hydroquinone, benzhydrol, methylhydrazine, 2-methyl-1,3-cyclopentanedione, acetylacetone, isopropyl alcohol, benzaldehyde, malononitrile or a combination thereof.

In one embodiment, the effective amount of the organic reductant, PHA, is sufficient to produce a concentration of 30 mM. In various embodiments, the effective amount of the reductant is sufficient to produce a concentration of 5-500 mM, 10-400 mM, 15-300 mM, 20-200 mM, or 25-100 mM.

In a further embodiment, the invention comprises exposing plasma to an effective amount of a carbon monoxide-producing molecule or carbon monoxide donor. In one embodiment, the carbon monoxide-producing molecule is tricarbonyldichlororuthenium (II) dimer ([RuCl2(CO)3]2; carbon monoxide releasing molecule-2; CORM-2). In various embodiments, the carbon monoxide-producing molecule is tricarbonyldichlororuthenium (II) dimer ([RuCl2(CO)3]2; carbon monoxide releasing molecule-2; CORM-2), iron pentacarbonyl (Fe(CO)₅), dimanganese decacarbonyl (Mn2(CO)10; CORM-1), tricarbonylchloro(glycinato)ruthenium (II) (Ru(CO)3Cl(glycinate); CORM-3), sodium boranocarbonate (Na2[H3BCO2]; CORM-A1), or a combination thereof.

In one embodiment, the effective amount of the carbon monoxide-producing molecule, CORM-2, is sufficient to produce a concentration of 100 μM. In various embodiments, the effective amount of the carbon monoxide-producing molecule is sufficient to produce a concentration of 5-600 μM, 10-500 μM, 15-400 μM, 20-300 μM, 25-200 μM or 30-100 μM.

In one embodiment, diagnosing hypocoagulability comprises detecting that the clot strength of the subject plasma is less than the clot strength of plasma obtained from a subject known to not have hypocoagulability. In one embodiment, diagnosing hypocoagulability comprises detecting that clot strength of the subject plasma is less than an institutionally generated 95% confidence interval.

In one embodiment, diagnosing hypocoagulability comprises detecting that the clot strength of the subject plasma does not substantially decrease upon exposure to an organic reductant (e.g. PHA). In one embodiment, hypocoagulability is determined by evaluating the PHA-mediated decrease in the clot strength of a sample, wherein the PHA-mediated decrease is given by the percent decrease in clot strength of PHA treated plasma compared to untreated plasma obtained from the same source. In one embodiment, hypocoagulability is diagnosed if the PHA treated plasma exhibits a clot strength that is not decreased by at least 55% compared to the subject's untreated plasma. In another embodiment, hyperoagulability is diagnosed if the PHA treated plasma exhibits a clot strength that is not decreased by at least 45% compared to the subject's untreated plasma. In another embodiment, hypocoagulability is diagnosed if the PHA treated plasma exhibits a clot strength that is not decreased by at least 35% compared to the subject's untreated plasma. In another embodiment, hypocoagulability is diagnosed if the PHA treated plasma exhibits a clot strength that is not decreased by at least 25% compared to the subject's untreated plasma. In another embodiment, hypocoagulability is diagnosed if the PHA treated plasma exhibits a clot strength that is not decreased by at least 10% compared to the subject's untreated plasma. In one embodiment, hypocoagulability is diagnosed if the PHA-mediated decrease in clot strength of a subject's plasma is less than the PHA-mediated decrease in clot strength of control plasma, obtained from a source known to not have hypocoagulability. In another embodiment, hypocoagulability is diagnosed if the PHA-mediated decrease in clot strength of a subject's plasma is less than an institutionally generated 95% confidence interval of PHA-mediated decreases in clot strength.

In one embodiment, diagnosing hypocoagulability comprises detecting that the clot strength of the subject plasma substantially increases upon exposure to carbon monoxide producing molecule (e.g. CORM-2). In one embodiment, hypocoagulability is determined by evaluating the CORM-2-mediated increase in the clot strength of a sample, wherein the CORM-2-mediated increase is given by the percent increase in clot strength of CORM-2 treated plasma compared to untreated plasma obtained from the same source. In one embodiment, hypocoagulability is diagnosed if the CORM-2 treated plasma exhibits a clot strength that is increased by at least 100% compared to the subject's untreated plasma. In another embodiment, hypocoagulability is diagnosed if the CORM-2 treated plasma exhibits a clot strength that is increased by at least 120% compared to the subject's untreated plasma. In another embodiment, hypocoagulability is diagnosed if the CORM-2 treated plasma exhibits a clot strength that is increased by at least 140% compared to the subject's untreated plasma. In another embodiment, hypocoagulability is diagnosed if the CORM-2 treated plasma exhibits a clot strength that is increased by at least 160% compared to the subject's untreated plasma.

A subject in need of assessment of the risk of developing hypocoagulability associated with nitric oxide exposure by the methods described herein includes any subject exposed or potentially exposed, acutely or chronically, to nitric oxide.

A subject in need of assessment of the risk of developing hypocoagulability includes those exposed or potentially exposed to exogenous nitric oxide. For example, subjects may be exposed to higher than normal nitric oxide exogenously present in their local environments. By way of non-limiting examples a subject in need of assessment of the risk of developing hypocoagulability associated with exposure to nitric oxide include, but are not limited to patients receiving chronic nitrates or patients needing acute or chronic nitric oxide inhalation.

Further, a subject in need of assessment of the risk of developing hypocoagulability includes those exposed or potentially exposed to endogenously produced nitric oxide. The sepsis, trauma, and critical care populations have been demonstrated to have both coagulopathy and increased circulating concentrations of methemoglobin (secondary to NO binding to hemoglobin) or the breakdown products of NO, nitrate or nitrite. Excessive nitric oxide production is known to contribute to various pathologic conditions including inflammatory conditions, neurodegeneration, heart disease, heart failure, and autoimmune disorders. Therefore, non-limiting examples of a subject in need of assessment of the risk of developing hypocoagulability associated with endogenous nitric oxide production includes trauma patients, critical care patients, septic patients, as well as those having or suspected to have an inflammatory disorder, autoimmune disorder, a neurodegenerative disease, heart disease, and heart failure.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Redox-Based Thromboelastic Method to Detect Carboxyhemefibrinogen-Mediated Hypercoagulability

### General Assay Design.

Pooled normal plasma (George King Bio-Medical, Overland Park, Kans., USA) anticoagulated with sodium citrate was utilized for experimentation. The lot of plasma used for all experimentation was #1853, with a prothrombin time of 12.3 sec, an activated partial thromboplastin time of 29.3 sec, and a fibrinogen concentration >250 mg/dl. The final volume for all subsequently described plasma sample mixtures was 359.2 μl. Sample composition consisted of 322 μl of plasma; 10 μl of tissue factor reagent (0.1% final concentration in dH₂O; Instrumentation Laboratory, Lexington, Mass., USA), 3.6 μl of dH₂O or dH₂O containing the organic reductant phenylhydroxylamine (PHA, 0-30 mM final concentration, Sigma-Aldrich, Saint Louis, Mo., USA), 3.6 μl of dimethyl sulfoxide (DMSO) or DMSO with CORM-2 (0-400 μM final concentration; Sigma-Aldrich) and 20 μl of 200 mM CaCl₂. PHA was chosen as the agent to convert fibrinogen-associated, heme-bound Fe⁺² to an Fe⁺³ state (1978, Castro et al., Biochemistry 17:225-231; 1986, Harrison and Jollow, J Pharmacol Exp Ther 238:1045-1054; 1987, Harrison and Jollow, Mol Pharmacol 32:423-431; 1999, Ciccoli et al., Toxicol Lett 110:57-66). Stock solutions of CORM-2 were made just before each experiment. Tissue factor stock solution was kept on ice prior to use and was remade every two hours.

Plasma sample mixtures were placed in a disposable cup in a computer-controlled thrombelastograph hemostasis system (Model 5000, Haemoscope Corp., Niles, Ill., USA), with addition of CaCl₂ as the last step to initiate clotting. Data were collected for 15 min at 37° C. Given that final clot strength is the sine quo non of fibrinogen concentration in plasma (2005, Nielsen et al., Acta Anaesthesiol Scand 49:222-231), and that elastic modulus (G, dyne/cm²) is a measure of strength that can be determined by all commercially available thrombelastographs/thromboelastometers, G values were subsequently recorded for all samples.

Additional elastic modulus-based parameters previously described were determined for some of the experiments (2009, Nielsen et al., Fibrinolysis 20: 377-380; Nielsen et al., in press, Fibrinolysis; 2005, Nielsen et al., Acta Anaesthesiol Scand 49: 222-231; 2009, Nielsen et al., Blood Coagul Fibrinolysis 20: 448-455). The nomenclature used to describe these phenomena is as follows:

Time to maximum rate of thrombus generation (TMRTG): This is the time interval (min) observed prior to maximum speed of clot growth.

Maximum rate of thrombus generation (MRTG): This is the maximum velocity of clot growth observed (dynes/cm²/sec).

Total Thrombus Generation (TTG): This is the total area under the velocity curve during clot growth (dynes/cm²), representing the amount of clot strength generated during clot growth.

### Statistical Analyses and Graphics.

Thrombelastographic data are presented as mean±SD. Analyses of the effects of PHA before and after CORM-2 addition to plasma were conducted with one way analysis of variance with Holm-Sidak post hoc test (SigmaStat 3.1, Systat Software, Inc., San Jose, Calif., USA). Comparison between plasma exposed to 10 or 30 mM PHA was performed with an unpaired Student's t-test. The plasmatic strength response to increasing concentrations of CORM-2 was analyzed with one way analysis of variance with Holm-Sidakpost hoc test. Graphical representation of the data was generated with commercially available software (Origin 7.5, OriginLab Corp., Northampton, Mass., USA). A P value of <0.05 was considered significant.

The results of the experiments are now described.

### Prevention of CO-Mediated Increases in Clotting and Clot Strength

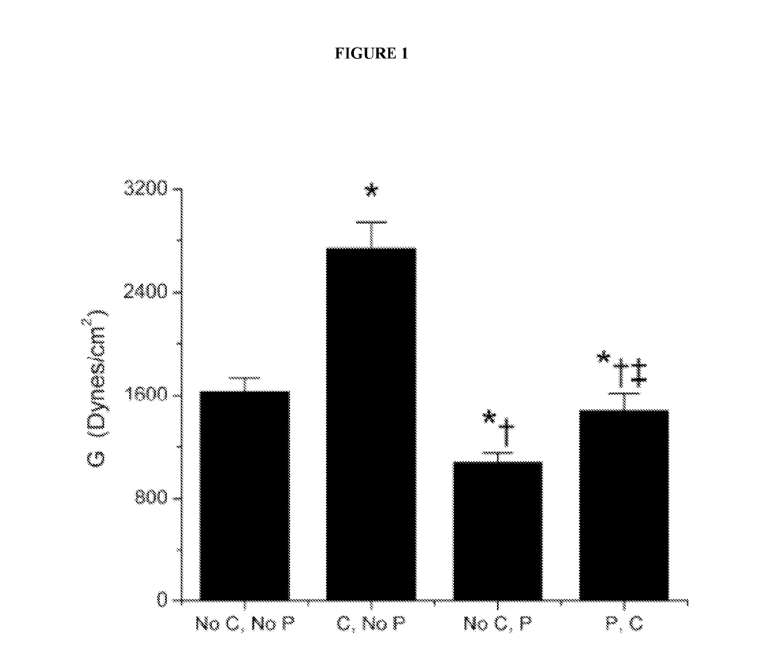

To determine if PHA could prevent/compete with CO binding to fibrinogen-bound heme, PHA at concentrations of 0, 1, 5 or 10 mM (n=4-8 experiments per condition) were added to plasma mixtures 2 min prior to addition of 100 μM CORM-2. As can be noted in FIG. 1, CORM-2 exposure significantly increased G values compared to unexposed plasma values (FIG. 1). Pretreatment with PHA at 1 or 5 mM concentrations had little effect on reducing CORM-2 mediated increases in G; however, exposing plasma to 10 mM PHA decreased G values in the presence of CORM-2 to values significantly smaller than plasma not exposed to either compound (FIG. 1). Lastly, exposure of plasma to 10 mM PHA alone also resulted in G values less than the other three conditions (FIG. 1).

With regard to the effects of PHA and CORM-2 on the other coagulation kinetic parameters, as displayed in Table 1, PHA addition significantly prolonged TMRTG compared to samples without additions or samples exposed to CORM-2. Addition of CORM-2 to samples already exposed to PHA had further significant prolongation of TMRTG greater than the other three conditions (Table 1). With regard to TMRTG, CORM-2 significantly increased the velocity of clot formation compared to plasma without additions; further, addition of PHA resulted in MRTG values significantly less than plasma with or without addition of CORM-2. However, addition of CORM-2 to plasma already exposed to PHA demonstrated MRTG values not different from plasma without additions (Table 1). The changes in TTG in response to addition of PHA and CORM-2 followed and identical pattern as demonstrated by MRTG (Table 1).

TABLE 1
Effects of CORM-2 and PHA on coagulation kinetics.

| Condition | TMRTG | MRTG | TTG |
|---|---|---|---|
| Control Plasma | 2.68 ± 0.51 | 6.2 ± 1.2 | 162 ± 10 |
| CORM-2 | 2.46 ± 0.15 | 12.4 ± 1.4* | 272 ± 21* |
| PHA 10 mM | 3.59 ± 0.60*† | 3.6 ± 0.9*† | 99 ± 11*† |
| PHA 10 mM then | 4.30 ± 0.62*†‡ | 5.3 ± 1.5†‡ | 168 ± 25†‡ |
| CORM-2 | | | |
| PHA 30 mM | 3.91 ± 0.70*† | 3.9 ± 0.8*† | 106 ± 8*† |
| CORM-2 then | 3.70 ± 0.30*† | 5.9 ± 0.7†# | 147 ± 14†# |
| PHA 30 mM | | | |

Data presented as mean ± SD.
Control Plasma = no additives,
CORM-2 = 100 μM CORM-2, other conditions are self-explanatory.
*P < 0.05 vs. Control Plasma;
†P < 0.05 vs. CORM-2;
‡P < 0.05 vs. PHA 10 mM;
#P < 0.05 vs. PHA 30 mM.

### Reduction of CO-Mediated Clotting and Clot Strength

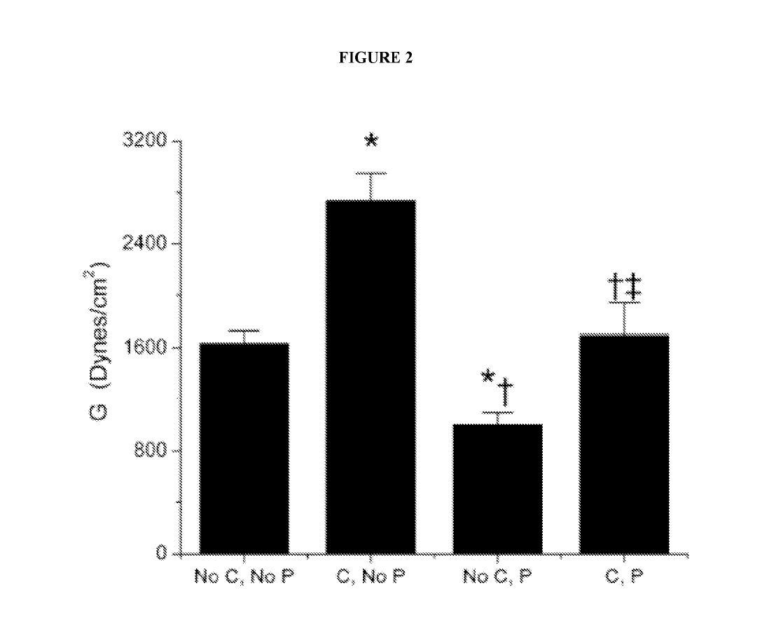

To determine if PHA could remove/compete with CO already in situ on fibrinogen-bound heme, 100 μM CORM-2 was added for 5 min prior to addition of 0, 10, 20 or 30 mM PHA (n=4-8 experiments per condition) to plasma mixtures. As depicted in FIG. 2, CORM-2 exposure significantly increased G values compared to unexposed plasma values. Following exposure to CORM-2, addition of 10 or 20 mM PHA had negligible effects on G (data not shown); however, addition of 30 mM PHA decreased G to values not different from plasma not exposed to either compound (FIG. 2). Lastly, the addition of 30 mM PHA alone resulted in G values significantly less than the other three conditions; however, these G values were not different than plasma exposed to 10 mM PHA as displayed in FIG. 1.

The effects of PHA addition following exposure to CORM-2 on the other coagulation kinetic parameters are also displayed in Table 1. While TMRTG values were significantly prolonged by addition of 30 mM PHA compared to plasma with no additions or addition of CORM-2, there was no significant difference in TMRTG values between plasma exposed to PHA or plasma first exposed to CORM-2 prior to PHA exposure (Table 1). With respect to MRTG, CORM-2 addition significantly increased MRTG compared to conditions without additions. PHA addition at 30 mM concentration significantly decreased MRTG compared to the other three conditions, whereas MRTG values in plasma first exposed to CORM-2 before PHA exposure had MRTG values not different from plasma without additions (Table 1). The pattern of TTG response in these experiments was identical to that of MRTG (Table 1).

### Inability for CO to Overcome PHA Mediated Reductions in Clot Strength

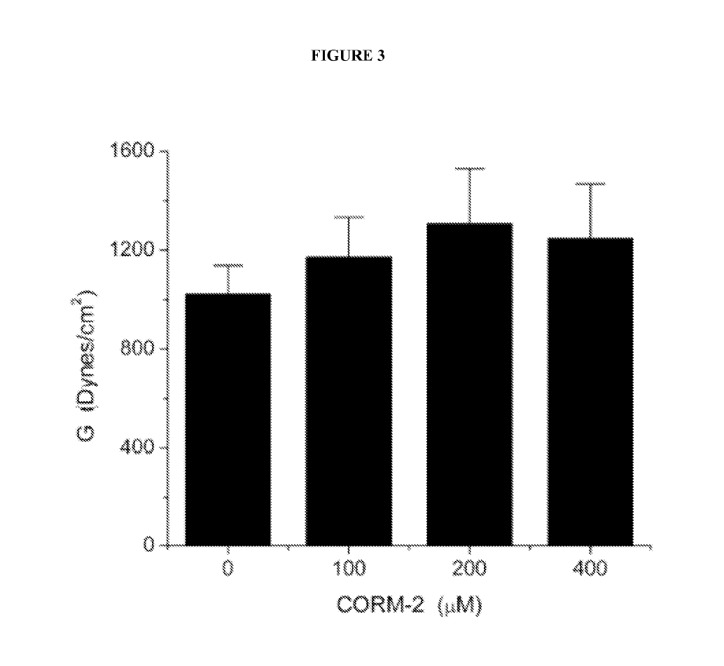

To determine if CO could outcompete PHA at the minimum concentration of PHA required to attenuate near maximum CO-mediated enhancement of G, plasma exposed to 30 mM PHA for 2 min was subsequently exposed to 0, 100, 200 or 400 μM CORM-2 for 5 min (n=4 per condition). As displayed in FIG. 3, plasma exposed to 30 mM PHA had no significant increase in G values in response to addition of 100, 200 or 400 μM CORM-2 (FIG. 3).

### Concentration-Response of Clot Strength in Response to CORM-2.

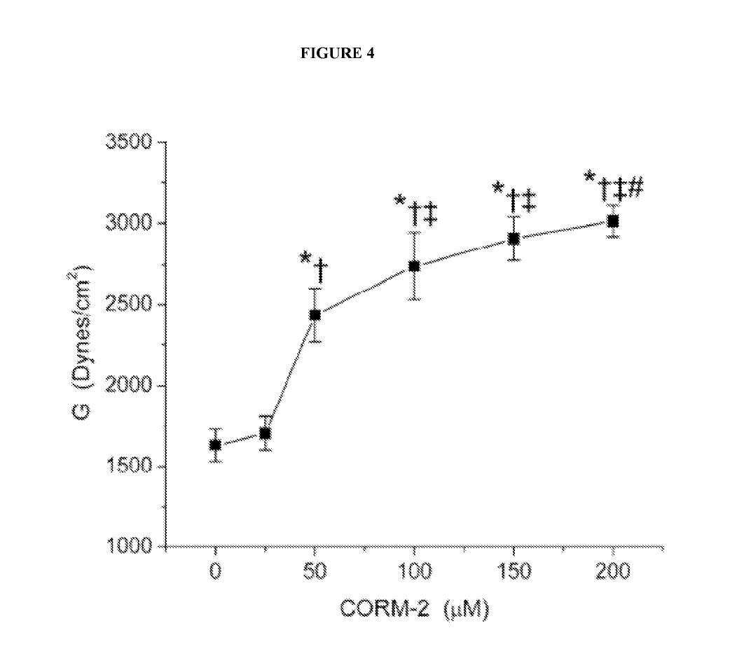

While a concentration of 100 μM CORM has been demonstrated to maximally enhance coagulation kinetics in previous investigations (2009, Nielsen et al., Blood Coagul Fibrinolysis 20:377-380; 2009, Nielsen et al., Blood Coagul Fibrinolysis 20:448-455), a more extensive concentration range was assessed in anticipation of utilizing addition of CORM-2 as a measure of saturable CO binding of fibrinogen-bound heme in the assessment of hypercoagulability. Plasma was exposed to 0, 25, 50, 100, 150 and 200 μM CORM-2 (n=8 per concentration) 5 min prior to commencement of coagulation in the absence of PHA. As displayed in FIG. 4, CORM-2 addition resulted in a sigmoidal increase in G value with increasing concentrations. Importantly, samples exposed to 100 and 150 μM CORM-2 did not have significantly increased G values. Lastly, while samples exposed to 200 μM CORM-2 had G values significantly greater than samples exposed to 100 μM CORM-2, the quantitative difference was only 10.1%.

### Diagnostic Assay

Given the results disclosed herein, a one-step, two-step and three-step approach to diagnosing hypercoagulability associated with carbon monoxide exposure is described. First, a subject's clot strength greater than an institutionally generated normal 95% confidence interval, is consistent with hypercoagulability associated with carbon monoxide exposure. Second, when a subject's plasma is exposed to an organic reductant, a decrease in the clot strength by more than about 40% (as compared with the subject's plasma not exposed to an organic reductant) is consistent with hypercoagulability associated with carbon monoxide exposure. Third, when a subject's plasma is exposed to a carbon monoxide-producing molecule, an increase in clot strength by less than about 35% (as compared with the subject's plasma not exposed to a carbon monoxide producing molecule), is consistent with hypercoagulability associated with carbon monoxide exposure. In various embodiments of the invention described herein, steps one, two or three can each be performed separately to reach a diagnosis, or each of steps one, two and three can be performed in combination with any of the other steps, in any order, to reach a diagnosis.

There are a variety of clinical scenarios wherein carboxyhemefibrinogen-mediated hypercoagulability may exist, where the described diagnostic method would be useful in detecting risk. First, smoking could inflict important hypercoagulability and the positive hemostatic effects of its cessation in the perioperative period could be verified with this assay. Further, evaluation of patients with carbon monoxide poisoning (e.g., firefighters, industrial workers) could be performed in resuscitative or perioperative settings to assess restoration of normal coagulation following oxygen therapy. Of note, chronic systemic diseases associated with hypercoagulability such as coronary artery disease (Idriss et al., 2010, Thromb Haemost, 104: 1029-1037), type 2 diabetes mellitus (Bao et al., 2010, PLoS One, 5: e12371), uremia treated with dialysis (Maroti et al., 2004, Pediatr Nephrol, 19: 426-431), and hepatic cirrhosis (De Las Heras et al., 2003, Hepatology, 38: 580-584) have been noted to involve either increased carbon monoxide production or heme oxygenase-1 (the endogenous source of carbon monoxide) upregulation. Lastly, ventricular assist devices, often associated with hypercoagulability and thromboembolic morbidity, are known to inflict various degrees of hemolysis, which would be expected to upregulate heme oxygenase-1 activity via increases in circulating heme (Heilmann et al., 2009, Eur J Cardiothorac Surg, 36: 580-584)[19]. In sum, it is likely that the proposed assay to detect carboxyhemefibrinogen-mediated hypercoagulopathy may be of use in acute care or ambulatory clinical settings.

In conclusion, using recent findings of a fibrinogenbound heme to explain carbon monoxide-mediated hypercoagulability, a redox-based method to detect and quantify potential thrombophilia secondary to carbon monoxide exposure has been created. This method can be used to assess the effects of smoking and environmental exposure to carbon monoxide on coagulation.

### Example 2

### Thrombelastographic Characterization of Coagulation/Fibrinolysis in Horses: Role of Carboxyheme and Metheme States

Carboxyheme and metheme states modulate hemostasis in humans and other species. Further, carbon monoxide (CO) and/or nitric oxide (NO) production increase in inflammatory disorders involving the gastrointestinal tract, with associated hypercoagulability or hypocoagulability. Since the observations were made that coagulation and fibrinolysis were modulated by CO in vitro (Nielsen et al., 2009, Blood Coagul Fibrinolysis, 20: 377-380; Nielsen et al., 2009, Blood Coagul Fibrinolysis, 20: 448-455) in human plasma and that CO and NO also modulate hemostasis in vitro and in vivo in rabbits (Nielsen et al., 2011, Blood Coagul Fibrinolysis, 22: 756-759; Nielsen et al., 2012, Blood Coagul Fibrinolysis, 23: 104-107; Nielsen et al., 2001, Anesth Analg, 92: 320-323), there has been an interest in comparing species-specific hemostatic responses to these two gases (Nielsen et al., 2009, Blood Coagul Fibrinolysis, 20: 377-380; Nielsen et al., 2009, Blood Coagul Fibrinolysis, 20: 448-455; Nielsen et al., 2011, Blood Coagul Fibrinolysis, 22: 756-759; Nielsen et al., 2012, Blood Coagul Fibrinolysis, 23: 104-107; Nielsen et al., 2001, Anesth Analg, 92: 320-323; Nielsen et al., 2010, Blood Coagul Fibrinolysis, 21: 298-299). Modulation of fibrinogen (Nielsen et al., 2011, Blood Coagul Fibrinolysis, 22: 657-661), plasmin (Arkebauer et al., 2011, Blood Coagul Fibrinolysis, 22: 712-719), and indirectly α₂-antiplasmin (Arkebauer et al., 2011, Blood Coagul Fibrinolysis, 22: 712-719) by CO and NO appear to be via binding to an attached heme(s) in human plasma. Both rabbit (Nielsen et al., 2011, Blood Coagul Fibrinolysis, 22: 756-759; Nielsen et al., 2012, Blood Coagul Fibrinolysis, 23: 104-107; Nielsen et al., 2001, Anesth Analg, 92: 320-323; Nielsen et al., 2010, Blood Coagul Fibrinolysis, 21: 298-299) and rat (Nielsen et al., 2010, Blood Coagul Fibrinolysis, 21: 298-299) plasma react kinetically to addition of CO or NO in a fashion similar to human plasma, supporting the presence of heme-based modulation. Identification of human analogues to investigate either CO-mediated hypercoagulability or NO-mediated hypocoagulation in disease states is critical in order to obtain further mechanistic insight and identify potential therapies. For example, intestinal inflammation has been associated with increased production of NO (Hanukoglu et al., 1996, J Pediatr Gastroenterol Nutr, 23: 1-7) and CO (Takagi et al., 2008, J Gastroenterol Hepatol, 23: S229-S233), with tendency towards thrombotic disease reported in human adults (Di Fabio et al., 2011, Semin Thromb Hemost, 37: 220-225; Murthy et al., 2011, Am J Gastroenterol, 106: 713-718; Kappelman et al., 2011, Gut, 60: 937-943) and children (Kappelman et al., 2011, Gut, 60: 937-943). Identification of an animal model of inflammatory intestinal disease with hemostatic responses to NO and CO similar to that of humans could be utilized to obtain a deeper understanding of human pathophysiology.

One such potential species is the horse, with both coagulopathic (Epstein et al., 2011, J Vet Intern Med, 25: 307-314) and thrombotic (Mendez-Angulo et al., 2011, Aust Vet J, 89: 500-505) courses noted after the onset of acute gastrointestinal disease of various etiologies. Typically, these horses are hypercoagulable on presentation, with progression to being hypocoagulable and hypofibrinolytic. In a recent study, 40% of horses with severe gastrointestinal disease had fibrin deposits in a number of tissues, indicating a systemic activation of coagulation (Epstein et al., 2011, J Vet Intern Med, 25: 307-314; Monreal et al., 2000, Equine Vet J Supplement, 32: 19-25; Cotovio et al., 2007, J Vet Intern Med, 21: 308-313). Of interest, the hemostatic state in two recent studies utilized thrombelastographic criteria (Epstein et al., 2011, J Vet Intern Med, 25: 307-314) and thrombotic (Mendez-Angulo et al., 2011, Aust Vet J, 89: 500-505); indeed, the viscoelastic characterizations of equines are quite recent and few in number (Paltrinieri et al., 2008, 37: 277-285; Leclere et al., 2009, Vet Clin Pathol, 38: 462-466; McMichael et al., 2011, Blood Coagul Fibrinolysis, 22: 424-430). Of interest, the horse has documented circulating plasmatic NO (50 nM) and CO (50 pM) (Maranon et al., 2008, Acta Veterinaria Scandinavica, 50: 45) that increase in response to stress such as jumping training (Leclere et al., 2009, Vet Clin Pathol, 38: 462-466). Taken as a whole, it is conceivable that intestinal inflammation and/or its associated stress may increase CO and/or NO, with the relative ratio of the gases modulating coagulation/fibrinolysis in the horse. Thus, with regard to changes in hemostasis, the horse model of enteritis could serve as an analogous model of intestinal inflammation in humans.

The studies presented herein characterize the coagulation/fibrinolytic responses during conditions favoring carboxyheme or metheme formation in normal horse plasma with thrombelastography as previously described (McMichael et al., 2011, Blood Coagul Fibrinolysis, 22: 424-430). Further, the analysis of samples obtained from a few horses with various degrees of enteritis is described to provide insight into potential heme-mediated changes in hemostasis.

The studies presented herein characterized the thrombelastographic response to carboxyheme (via CORM-2) or metheme (via phenylhydroxylamine, PHA) states without/with addition of tissue type plasminogen activator. Citrated plasma was obtained from 14 normal mares and 3 horses with enteritis. In normal horses, a carboxyheme state did not enhance the velocity of clot growth and minimally enhanced clot strength (9%). In contrast, a metheme state was associated with a decrease in the velocity of clot formation (54%) and clot strength (47%).

During fibrinolysis, a carboxyheme state significantly decreased the onset (113%) and velocity (27%) of fibrinolysis; however, in contrast, a metheme state more markedly increased the onset (84%) and velocity (133%) of fibrinolysis. These data support a CO-dominant modulation of hemostasis in normal horses. In contrast, an increase in the severity of acute gastrointestinal disease was associated with a likely NO-mediated, metheme state-induced hypocoagulable/hyperfibrinolytic state.

The materials and methods employed are now described.

Plasma Collection and Processing.

Blood was collected directly into plastic tubes containing 0.105 M sodium citrate (9:1, v/v) from the jugular vein of ten clinically normal grade and American Quarter Horse mares between 400 and 600 kg. Three additional horses to be subsequently described with acute gastrointestinal disease had blood samples similarly collected. The mares were group-housed in corrals. Plasma was obtained by centrifugation of citrated whole blood at 3000×g for 15 minutes and immediately frozen at −80° C. Plasma was subsequently shipped on dry ice and stored at −80° C. until thrombelastographic analysis.

Coagulation Study Plasma Mixtures.

The final volume for all subsequently described plasma sample mixtures was 359.6 μl. Sample composition for this series of experiments consisted of 326 μl of plasma; 10 μl of tissue factor reagent (0.1% final concentration in dH₂O; Diagnostica Stago S.A.S., Asnieres sur Seine, France); 3.6 μl of dH₂O, or 3.6 μl dH₂O containing the heme-binding reductant phenylhydroxylamine (PHA, 10 mM final concentration, Sigma-Aldrich, Saint Louis, Mo., USA), or 3.6 μl of dimethyl sulfoxide with CORM-2 (100 μM final concentration; Sigma-Aldrich, Saint Louis, Mo., USA); and 20 μl of 200 mM CaCl₂. As previously described in human plasma (McMichael et al., 2011, Blood Coagul Fibrinolysis, 22: 424-430), this diagnostic panel is designed to determine hemostatic kinetic changes following of formation of carboxyhemefibrinogen (Fe⁺² state) after exposure to CO released from CORM-2 and to determine the effects of formation of methemefibrinogen (Fe⁺³ state) after exposure to PHA. The relative change from baseline kinetics caused by exposure to CORM-2 and PHA allow determination of the balance between oxy/carboxy/methemefibrinogen concentrations in human plasma (McMichael et al., 2011, Blood Coagul Fibrinolysis, 22: 424-430).

Coagulation and Fibrinolysis Study Plasma Mixtures.

In the second series of experiments assessing both coagulation and fibrinolysis, sample composition consisted of 316 μl of plasma; 10 μl of tissue factor reagent (0.1% final concentration), 10 μl of tissue type plasminogen activator (tPA, 580 Genentech, Inc., San Francisco, Calif., USA; 300 IU/ml final concentration); 3.6 μl of dH₂O, or 3.6 μl dH₂O containing PHA (10 mM final concentration), or 3.6 μl of CORM-2 (100 μM final concentration) and 20 μl of 200 mM CaCl₂. The effects of converting heme groups to either a carboxyheme or metheme in horse plasma during clot growth and fibrinolysis were assessed as subsequently described.

Thrombelastographic Analyses.

Plasma sample mixtures were placed in a disposable cup in a computer-controlled Thrombelastograph® hemostasis system (Model 5000, Haemoscope Corp., Niles, Ill., USA), with addition of CaCl₂ as the last step to initiate clotting. Data were collected at 37° C. for 15 min in the first series of experiments involving coagulation alone, whereas in the experiments involving fibrinolysis data were collected until clot lysis time was observed. Elastic modulus-based parameters previously described were determined (Nielsen et al., 2009, Blood Coagul Fibrinolysis, 20: 377-380; Nielsen et al., 2009, Blood Coagul Fibrinolysis, 20: 448-455). The nomenclature used to describe these phenomena is as follows:

Time to maximum rate of thrombus generation (TMRTG): This is the time interval (min) observed prior to maximum speed of clot growth.

Maximum rate of thrombus generation (MRTG): This is the maximum velocity of clot growth observed (dynes/cm²/sec).

Total Thrombus Generation (TTG): This is the total area under the velocity curve during clot growth (dynes/cm²), representing the amount of clot strength generated during clot growth.

Time to maximum rate of lysis (TMRL): This is the time interval (min) measured from the time of maximum strength to the time when the velocity of clot disintegration is maximal.

Maximum Rate of Lysis (MRL): This is the maximum velocity of clot disintegration observed (-dynes/cm²/sec).

Clot growth time (CGT): This is the time from clot amplitude of 2 mm [102 dynes/cm²] until maximum strength is achieved, in min.

Clot lysis time (CLT): This is the time from when maximum strength was observed to 2 mm amplitude, in min.

Clot lifespan (CLS): This is the sum of CGT and CLT.

Statistical Analyses.

Thrombelastographic data are presented as mean±SD or as individual animal parameter values as appropriate. The effects of addition of CORM-2 and PHA on coagulation and fibrinolytic kinetic parameters were analyzed with one way repeated measures analysis of variance, utilizing Holm-Sidak post hoc test (SigmaStat 3.1, Systat Software, Inc., San Jose, Calif., USA) as appropriate. A P value of <0.05 was considered significant.

The results of the experiments are now described.

### Effects of CORM-2 and PHA on Coagulation Kinetics.

The effects of establishing a carboxyheme or metheme state in horse plasma are displayed in Table 2. Both CORM-2 and PHA addition significantly prolonged R values compared to samples without additions. CORM-2 did not significantly affect a values, but PHA addition resulted in a values significantly different from either control or CORM-2 exposed conditions. The MA values of CORM-2 exposed samples were significantly, but only mildly increased compared to control conditions; in contrast, MA was significantly less in PHA exposed samples compared to the other two conditions. Similar to changes in R, TMRTG values were prolonged by both CORM-2 and PHA. As with a, CORM-2 did not significantly change MRTG compared to control conditions, whereas exposure to PHA significantly decreased (54%) MRTG values compared to the other two conditions. Lastly, a significant but small increase (9%) in TTG after exposure to CORM-2 compared to control conditions, whereas a significant decrease (47%) in TTG values compared to control values was noted after PHA addition.

TABLE 2
Effects of CORM-2 and PHA on coagulation kinetics

| Condition | R | α | MA | TMRTG | MRTG | TTG |
|---|---|---|---|---|---|---|
| Control | 1.6 ± 0.3 | 80.5 ± 2.1 | 38.0 ± 3.7 | 2.4 ± 0.4 | 13.4 ± 3.1 | 313 ± 48 |
| CORM-2 | 2.0 ± 0.4* | 80.2 ± 2.1 | 40.4 ± 3.4* | 2.8 ± 0.5* | 13.0 ± 2.9 | 342 ± 48* |
| PHA | 2.4 ± 0.4*† | 71.7 ± 3.6*† | 24.6 ± 2.7*† | 3.4 ± 0.6*† | 6.2 ± 1.7*† | 166 ± 23*† |

Data presented as mean ± SD.
Control = no additives,
CORM-2 = plasma with 100 μM CORM-2,
PHA = plasma with 10 mM phenylhydroxylamine.
*P < 0.05 vs. Control;
†P < 0.05 vs. CORM-2.

### Effects of CORM-2 and PHA on Coagulation/Fibrinolysis Kinetics.

When plasma was exposed to CORM-2 or PHA in the presence of tPA, R, α and MA values changed in an identical fashion compared to samples not exposed to tPA. Additional coagulation/fibrinolysis data derived from this series of experiments are displayed in Table 3. Both CORM-2 and PHA addition significantly increased TMRTG values compared to control samples. MRTG values were not significantly changed by CORM-2 addition when compared to control conditions; however, a significant decrease (55%) was observed following PHA addition compared to control sample values. TTG values were increased significantly, and to a greater extent than in the first series of experiments (35%) in samples exposed to CORM-2. Exposure to PHA significantly decreased (49%) TTG values compared to control conditions. With regard to the onset of lysis, TMRL values were prolonged significantly (113%) by exposure to CORM-2 whereas exposure to PHA decreased TMRL values significantly and markedly (84%) compared to control values. As for the velocity of lysis, CORM-2 exposure significantly decreased (27%) MRL values while PHA exposure significantly (133%) increased MRL values compared to control values. Significant increases in CGT (106%), CLT (160%) and CLS (154%) values were observed following exposure to CORM-2 when compared to control sample values. In contrast, significant decreases in CGT (26%), CLT (83%) and CLS (77%) values were observed following exposure to CORM-2 when compared to control sample values.

TABLE 3
Effects of CORM-2 and PHA on coagulation/fibrinolysis kinetics

| Condition | TMRTG | MRTG | TTG | TMRL | MRL | CGT | CLT | CLS |
|---|---|---|---|---|---|---|---|---|
| Control | 2.3 ± 0.3 | 12.5 ± 3.1 | 231 ± 43 | 12.3 ± 9.0 | 1.5 ± 0.5 | 3.1 ± 0.9 | 27.2 ± 14.1 | 30.3 ± 14.4 |
| CORM-2 | 2.7 ± 0.4* | 13.2 ± 3.4 | 312 ± 70* | 26.2 ± 17.3* | 1.1 ± 0.5* | 6.4 ± 1.7* | 70.6 ± 23.0* | 77.1 ± 23.8* |
| PHA | 3.2 ± 0.6*† | 5.6 ± 1.3*† | 118 ± 26*† | 2.0 ± 0.2*† | 3.5 ± 0.8*† | 2.3 ± 0.5 | 4.6 ± 1.3*† | 6.9 ± 1.4*† |

Data presented as mean ± SD.
Control = no additives,
CORM-2 = plasma with 100 μM CORM-2,
PHA = plasma with 10 mM phenylhydroxylamine.
*P < 0.05 vs. Control;
†P < 0.05 vs. CORM-2.

Characterization of Three Horses with Different Degrees of Acute Gastrointestinal Disease.

Three cases of horses presenting with enteritis that resulted in clinically mild, moderate and lethal severity are subsequently presented.

Mild Enteritis.

An 11 year old male, castrated quarter horse presented with an impacted colon. H is clinical data are presented in Table 4. He was mildly anemic, hyperfibrinogenemic, and had mildly prolonged activated partial thromboplastin time (aPTT) values. H is prothrombin time (PT) was normal. This horse recovered quickly.

Citrated plasma was obtained and analyzed as previously described, with results presented in Table 5. In the absence of tPA, addition of PHA or CORM-2 prolonged TMRTG. The MRTG value of unexposed plasma was markedly decreased compared to normal horses (Table 2), with a 45% decrease in MRTG noted after PHA addition which contrasted with the 16% increase in MRTG following CORM-2 addition. With regard to clot strength, this horse's TTG value was below normal, and PHA addition decreased TTG by 28% whereas CORM-2 exposure increased strength by 25%. In the presence of tPA, a similar pattern of decreased growth velocity and strength compared to normal values was noted. PHA addition decreased MRTG (42%) and TTG (28%), whereas CORM-2 exposure slightly decreased MRTG (3%) and increased TTG (77%) to twice the extent seen with normal horses. Of great interest, TMRL was very brief compared to normal horse values, not very different from normal horse plasma exposed to PHA, and finally only 8% different from the PHA exposed sample. Remarkably, CORM-2 addition increased TMRL 46-fold. MRL was increased by PHA (53%), whereas CORM-2 addition decreased MRL by 88%, more than 3 times greater change than that seen in normal plasma. Lastly, while control and PHA exposed samples had complete lysis, the CORM-2 exposed sample did not completely lyse by 191 min, with nearly half of the original clot strength remaining.

Moderate Enteritis.

The second case was a 16 year old Morgan mare that presented with watery diarrhea and loss of appetite. The working diagnosis was bacterial enteritis from which this horse quickly recovered. Clinical data are in Table 4. This horse had only a mildly prolonged aPTT and normal PT and fibrinogen concentration.

With regard to thrombelastographic data displayed in Table 5, the control sample without tPA addition demonstrated a velocity of clot growth and strength almost half of that of normal horses. PHA exposure decreased MRTG and TTG by 45% and 22%, respectively. The decrease in TTG was less than half of that observed with normal horse plasma. Of interest, CORM-2 addition increased MRTG and TTG by 28% and 47%, far greater increases than that observed in normal horse plasma. In the presence of tPA, control samples demonstrated nearly a two-third decrease in velocity of clot growth and strength compared to normal horse plasma. Remarkably, with addition of PHA, MRTG only decreased by 13% and TTG paradoxically increased by 23%. In contrast, CORM-2 exposure increased MRTG 38% and TTG by 160%. The control sample TMRL value was similar to that observed in PHA exposed normal plasma, and exposure of this sample to PHA resulted in a paradoxical 64% increase in TMRL. PHA exposure resulted in essentially no change in MRL compared to the control sample. In contrast, CORM-2 addition increased TMRL 55-fold and decreased MRL by 83%. Lastly, while control and PHA exposed samples had complete lysis, CORM-2 addition resulted in incomplete lysis, with nearly 75% of original clot strength remaining after 191 min.

Lethal Enteritis.

The last horse to be presented was a 10 year old castrated male Quarter horse that suffered colonic torsion, resection and anastomosis two days before laboratory data displayed in Tables 4 and 5 were obtained. Also, hydroxyethyl starch was administered the day before sampling was performed. This horse had marked prolongation of PT and aPTT with normal fibrinogen concentration. Despite maximum medical management, this horse was moribund 2 days after the time of sampling and was humanely euthanized.

Thrombelastographic data of this horse are displayed in Table 5. Control samples not exposed to tPA had approximately 20-25% of normal plasma clot growth velocity and strength. Exposure to PHA essentially extinguished coagulation, whereas addition of CORM-2 increased MRTG by 67% and TTG by 97%. Similar abysmal coagulation was noted in the control sample exposed to tPA, and given the previous response to PHA, this sample was not tested in anticipation of essentially no coagulation when in the fibrinolytic state. CORM-2 exposure increased MRTG and TTG by 12% and 73%, respectively. TMRL was near normal values in the control sample, but MRL was drastically reduced with final clot lysis occurring after 111 min. Exposure to CORM-2 increased TMRL 8-fold but did not affect MRL. However, complete clot lysis did not occur after 190 min, with one third of the original clot strength remaining.

TABLE 4
Clinical and Thrombelastographic Data of Ill Horses
Severity of Gastrointestinal Disease

| | Normal | | Moderate | Lethal |
|---|---|---|---|---|
| Clinical Data | Range | Mild Disease | Disease | Disease |
| Hematocrit (%) | (31-47%) | 26% | 36% | 35% |
| PT (sec) | (9.0-12.0) | 10.5 | 10.8 | 15.4 |
| aPTT (sec) | (26.0-52.0) | 53.8 | 58.0 | 92.0 |
| Fibrinogen | (100-400) | 500 | 200 | 400 |
| (mg/dl) | | | | |

PT = prothrombin time;
aPTT = activated partial thromboplastin time.

TABLE 5
Thrombelastographic Data of Ill Horses
Severity of Gastrointestinal Disease

| | Mild Disease | | | Moderate Disease | | | Lethal Disease | | |
|---|---|---|---|---|---|---|---|---|---|
| Condition | Con | PHA | CORM | Con | PHA | CORM | Con | PHA | CORM |
| No tPA | | | | | | | | | |
| TMRTG | 2.6 | 3.9 | 2.9 | 2.1 | 3.5 | 2.4 | 3.0 | 8.2 | 3.7 |
| MRTG | 8.8 | 4.8 | 10.2 | 7.1 | 3.9 | 9.1 | 3.0 | 0.1 | 5.0 |
| TTG | 247 | 177 | 308 | 162 | 126 | 238 | 64 | 2 | 126 |
| tPA | | | | | | | | | |
| TMRTG | 2.8 | 3.4 | 3.2 | 2.2 | 2.8 | 2.5 | 3.3 | — | 4.1 |
| MRTG | 7.0 | 4.0 | 6.8 | 5.2 | 4.5 | 7.2 | 2.5 | — | 2.8 |
| TTG | 155 | 100 | 274 | 76 | 94 | 198 | 53 | — | 92 |
| TMRL | 2.6 | 2.4 | 122.8 | 1.7 | 2.8 | 62.9 | 8.2 | — | 65.2 |
| MRL | 1.7 | 2.6 | 0.2 | 2.4 | 2.3 | 0.4 | 0.1 | — | 0.1 |
| % TTG | 100 | 100 | 50.7 | 100 | 100 | 25.8 | 100 | — | 67.4 |
| Loss | | | | | | | | | |

Con = control condition, no additives;
PHA = phenylhydroxylamine added, 10 mM final concentration;
CORM = CORM-2 added, 100 μM final concentration.

### Coagualtion and Fibrinolysis in Normal and Diseased Horses

The primary finding of the present study is that horse plasma coagulation and fibrinolysis are modulated by conditions that can be characterized as carboxyheme or metheme dominant states. Given the complexity of the present findings, it would be helpful to consider the normal horse plasma data first and then contrast them with those obtained from horses with enteritis.

Normal Horses.

First, it should be noted that horse plasma thrombi grow about twice as fast and are twice as strong as human (Nielsen et al., 2009, Blood Coagul Fibrinolysis, 20: 377-380) plasma clots and approximately 20% faster growing and stronger than rabbit plasma (Nielsen et al., 2011, Blood Coagul Fibrinolysis, 22: 756-759) clots. Second, despite the fact that there appears to be 3 orders of magnitude more NO than CO in the circulation of horses (Leclere et al., 2009, Vet Clin Pathol, 38: 462-466), it appears that coagulation is predominantly influenced by CO in the resting state (Table 2). This contention is supported by the relative lack of effect of CORM-2 on either the velocity of clot growth or strength—no change in MRTG and only 9% increase in TTG. This is markedly different from humans (Nielsen et al., 2009, Blood Coagul Fibrinolysis, 20: 377-380; Nielsen et al., 2009, Blood Coagul Fibrinolysis, 20: 448-455) or rabbits (Nielsen et al., 2011, Blood Coagul Fibrinolysis, 22: 756-759; Nielsen et al., 2012, Blood Coagul Fibrinolysis, 23: 104-107), wherein 100 μM CORM-2 will increase MRTG by 64-100% and TTG by 51-68%. Further, exposure to PHA decreases MRTG and TTG values (Table 2) in normal horse plasma to a greater extent than in human (Arkebauer et al., 2011, Blood Coagul Fibrinolysis, 22: 712-719) or rabbit (Nielsen et al., 2012, Blood Coagul Fibrinolysis, 23: 104-107) plasma. In sum, given the lack of change in coagulation kinetics in response to CO and the marked decrease in coagulation in response to a metheme state, it is likely that horse plasma coagulation is primarily CO dominated.

With regard to fibrinolysis, the horse requires 10-fold the concentration of tPA to induce fibrinolysis compared to humans (Nielsen et al., 2009, Blood Coagul Fibrinolysis, 20: 448-455), as has been observed with rats (Nielsen et al., 2010, Blood Coagul Fibrinolysis, 21: 298-299). The effects of carboxyheme and metheme states in the fibrinolytic environment are somewhat different, but it is still likely that CO dominates fibrinolytic processes in the horse. MRTG values are not affected by CORM-2, but instead are precipitously decreased following exposure to PHA (Table 3). TTG is increased by 35% by CORM-2, while PHA exposure decreases clot strength by 55%. This is markedly different from human plasma, wherein MRTG and TTG are increased 120 and 113% by CORM-2, respectively, and PHA decreases MRTG and TTG by 30% and 21%, respectively (Arkebauer et al., 2011, Blood Coagul Fibrinolysis, 22: 712-719). With regard to lysis, while CORM-2 prolonged the onset and speed of lysis so as to essentially double clot lifespan (Table 3), the marked and rapid onset of rapid lysis in the presence of PHA resulted in a clot lifespan reduction of 77% in horse plasma. In sum, as with coagulation, it appears that fibrinolysis is primarily modulated by CO, given the magnitude of enhanced fibrinolysis observed during the metheme state induced by PHA.

Horses with Enteritis.

The spectrum of disease severity provided important insight into the pathogenesis of coagulopathy in the setting of gastrointestinal disease. In the case of the horse with mild disease, slightly increased aPTT and hyperfibrinogemia, an important decrease in speed of clot formation (tissue factor initiated) and strength was observed thrombelastographically (Table 4) in the control sample without tPA. This is not what would be expected, given that hyperfibrinogemia was present. Further, a decreased response to PHA and increased response to CORM-2 compared to normal plasma samples likely indicate that NO, and not CO, is primarily modulating coagulation in this horse. With regard to fibrinolysis, enteritis appears to inflict a hyperfibrinolytic state in the first two horses, evidenced by hypocoagulation and rapid onset of lysis not increased by addition of PHA. Instead, exposure to CO via CORM-2 results in a far greater than normal hypofibrinolytic response. Indeed, the horse with moderate disease demonstrates a more progressive decrease in response to PHA and increased response to CORM-2. Taken as a whole, the first two horses appear to present a pattern of less CO modulation and more NO modulation of coagulation and fibrinolysis in proportion to the severity of disease present.

In contrast to the two other horses, the horse with lethal disease suffered multiple insults including sepsis, hemodilution with starch, and presumed consumptive coagulopathy. Unfortunately, PHA could not be used diagnostically secondary to limitations of this model; if functional fibrinogen concentrations are between 50-75 mg/dl, thrombelastographic data cannot be detected reliably (Nielsen et al., 2005, Acta Anaesthesiol Scand 2005, 49: 222-231). Thus, while fibrinogen concentration was normal (Table 4), increased NO, interference from hydroxyethyl starch and decreased thrombin generation secondary to loss of proximate coagulation factors (evidenced by prolonged PT and aPTT) likely resulted in very weak clot strength. NO predominance over coagulation in this animal is strongly indirectly supported by abnormal increases in velocity of clot growth and strength, prolongation of the onset of lysis and finally incomplete lysis after exposure to CORM-2. It is important to note that baseline hypofibrinolysis, not hyperfibrinolysis, was present in this animal. While not measured, it is speculated that a consumptive loss of plasminogen (rather than excess α₂-antiplasmin secretion) is responsible for this hypofibrinolytic state.

The studies presented herein have demonstrated that the coagulation and fibrinolytic systems of the horse respond to both carboxyheme and metheme states, albeit to differing extents compared to humans or rabbits. In the resting state, CO appears to predominate heme-based hemostasis, whereas NO appears to be a major player in mediating coagulopathy associated with enteritis.

### Example 3

### Study of Hypercoagulability and Carboxyhemefibrinogen Formation in Smoking and Normal Subjects

Tobacco Smoking and Thrombotic Disease.

Virtually every disease state associated with chronic or acute thrombosis (e.g., myocardial infarction, stroke, PVD) has had smoking identified as a risk factor (Agarwal, 2009, Angiology, 60: 335-345; Bhatt et al., 2006, JAMA, 295: 180-189; shah et al., 2010, Expert Rev Cardiovasc Ther., 8: 917-932; Oliveria et al., 2007, Preventative Medicine, 44: 311-316; Herman et al., 2011, Am J Public Health, 101: 491-496). The smoking-induced systemic hypercoagulable state involves an increase in circulating fibrinogen (Lind et al., 2004, Arterioscler Thromb Vasc Biol, 24: 577-582) that abates with cessation of smoking (Haustein et al., 2004, Int J Clin Pharacol Ther, 42: 83-92), an increase in circulating factor XIII (FXIII) (Ariens et al., 1999, Arteriol Thromb Vasc Biol, 19: 2012-2016; van Wersch et al., 1997, Int J Clin Lab Res, 27: 68-71), and enhanced activation of circulating platelets (Neubauer et al., 2009, Blood Coagul Fibrinolysis, 20: 694-698) that also decreases with smoking abatement (Morita et al., 2005, J Am Coll Cardiol, 45: 589-594). With regard to changes in viscoelastic properties, it has been demonstrated that both platelet and plasma mediated whole blood clot strength increased after quickly smoking two cigarettes (Barua et al., 2010, Arterioscler Thromb Vasc Biol, 30: 75-79), and vulnerability to clot lysis by tPA was also reduced (Barua et al., 2010, Thromb Res, 126: 426-430). Surprisingly, the increase in platelet or plasma mediated strength was only approximately 7% (Barua et al., 2010, Arterioscler Thromb Vasc Biol, 30: 75-79; Barua et al., 2010, Thromb Res, 126: 426-430). Of interest, scanning electron microscopy documented that fibrin polymer formations within clot formed after smoking had significantly thinner, denser fibers composing the matrix, which would on a physicochemical level explain increased clot strength and decreased vulnerability to clot lysis (Barua et al., 2010, Arterioscler Thromb Vasc Biol, 30: 75-79; Barua et al., 2010, Thromb Res, 126: 426-430). While no particular inhaled product of combustion was implicated by any of these studies (Lind et al., 2004, Arterioscler Thromb Vasc Biol, 24: 577-582; Haustein et al., 2004, Int J Clin Pharacol Ther, 42: 83-92; Ariens et al., 1999, Arteriol Thromb Vasc Biol, 19: 2012-2016; van Wersch et al., 1997, Int J Clin Lab Res, 27: 68-71; Neubauer et al., 2009, Blood Coagul Fibrinolysis, 20: 694-698; Morita et al., 2005, J Am Coll Cardiol, 45: 589-594; Barua et al., 2010, Arterioscler Thromb Vasc Biol, 30: 75-79; Barua et al., 2010, Thromb Res, 126: 426-430), expired or circulating concentrations of CO in smokers was associated with an increased incidence of adverse cardiovascular events (Hedblad et al., 2005, Atherosclerosis, 179: 177-183). Further, in never smokers, increased carboxyhemoglobin (COHb) concentrations (approximately 1% or greater) secondary to environmental exposure was associated with an increased incidence of cardiovascular morbidity (Hedblad et al., 2006, Scand J Public Health, 34: 609-615). Taken as a whole, smoking increases coagulation and attenuates fibrinolysis, and one of the products of cigarette smoking, CO, appears to be closely associated with adverse cardiovascular events.

CO Exposure Via CORM-2 Enhances Speed of Clot Formation, Clot Strength, and Decreases Fibrinolytic Vulnerability In Vitro and In Vivo.

A serendipitous discovery nearly three years ago revealed that CO released from CORM-2 enhanced plasmatic coagulation (Nielsen et al., 2009, Blood Coagul Fibrinolysis, 20: 377-380) and diminished fibrinolytic vulnerability to tPA as assessed by thrombelastography (Nielsen et al., 2009, Blood Coagul Fibrinolysis, 20: 448-455). This CO mediated enhancement of coagulation was independent of thrombin generation (Nielsen et al., 2010, Blood Coagul Fibrinolysis, 21: 349-353), did not involve FXIII (Nielsen et al., 2009, Blood Coagul Fibrinolyis, 20: 377-380; Nielsen et al., 2010, Blood Coagul Fibrinolysis, 21: 101-105), and seemed to directly affect fibrinogen (Nielsen et al., 2010, Blood Coagul Fibrinolysis, 21: 349-353; Machovec et al., 2012, Thromb Res, 129: 793-796). Further, transmission electron microscopy revealed a loss of thick fibrin polymer fibers and an increased density of thin fibers in thrombi formed from normal and FXIII-deficient plasma exposed to CORM-2 (Nielsen et al., 2010, Blood Coagul Fibrinolysis, 21: 101-105). To elucidate if CO modified fibrinogen directly, CORM-2 naïve and exposed purified fibrinogen were subjected to liquid chromatography-mass spectrometry, discovering no direct modifications but instead differences in vulnerability to protease digestion mediated by CO and the discovery of a previously undescribed attached heme group (Nielsen et al., 2011, Blood Coagul Fibrinolysis, 22: 443-447). The substrate properties of fibrinogen were enhanced by exposure to CO (Nielsen et al., 2010, Blood Coagul Fibrinolysis, 21: 349-353; Machovec et al., 2012, Thromb Res, 129: 793-796; Nielsen et al., 2011, Blood Coagul Fibrinolysis, 22: 443-447; Nielsen et al., 2011, Blood Coagul Fibrinolysis, 22: 657-661) and diminished by agents promoting a metheme (Fe⁺³) state (e.g., nitric oxide (NO), phenylhydroxylamine (PHA)) of heme groups (Nielsen et al., 2011, Blood Coagul Fibrinolysis, 22: 443-447; Nielsen et al., 2011, Blood Coagul Fibrinolysis, 22: 657-661). These findings were exploited to create a redox-based, thrombelastographic method to detect carboxyhemefibrinogen (COHF) and assess its contribution to CO associated hypercoagulability (Nielsen et al., 2011, Blood Coagul Fibrinolysis, 22: 657-661).

In vivo validation of the role of CO in hemostasis was subsequently achieved with a conscious rabbit model that utilized ear bleeding time (BT) as a primary endpoint. Rabbits were subjected to platelet inhibition with aspirin and clopidogrel (Nielsen et al., 2011, Blood Coagul Fibrinolysis, 22: 756-759) or administered intravenous tPA (Nielsen et al, 2012, Blood Coagul Fibrinolysis, 23: 104-107); in both models, injection with CORM-2 intravenously resulted in normal (Nielsen et al., 2011, Blood Coagul Fibrinolysis, 22: 756-759) or near normal (Nielsen et al., Blood Coagul Fibrinolysis, 2012, 23:104-107) bleeding times despite the iatrogenic coagulopathy.

With regard to CO mediated hypofibrinolysis, it was determined that α₂-antiplasmin (Malayaman et al., 2011, Blood Coagul Fibrinolysis, 22: 345-348; Arkebauer et al., 2011, Blood Coagul Fibrinolysis, 22: 712-719) and plasmin (Arkebauer et al., 2011, Blood Coagul Fibrinolysis, 22: 712-719) were affected by CO; specifically, CO enhanced α₂-antiplasmin activity whereas CO decreased plasmin activity. Further, plasmin was determined to have an attached heme group via liquid chromatography-mass spectrometry (Arkebauer et al., 2011, Blood Coagul Fibrinolysis, 22: 712-719); due to glycosylation of the enzyme, an attached heme on 122-antiplasmin was not able to be definitively demonstrated with this method.

Plasma Obtained from Young Smokers without Thrombotic Disease is Hypercoagulable and has COHF.

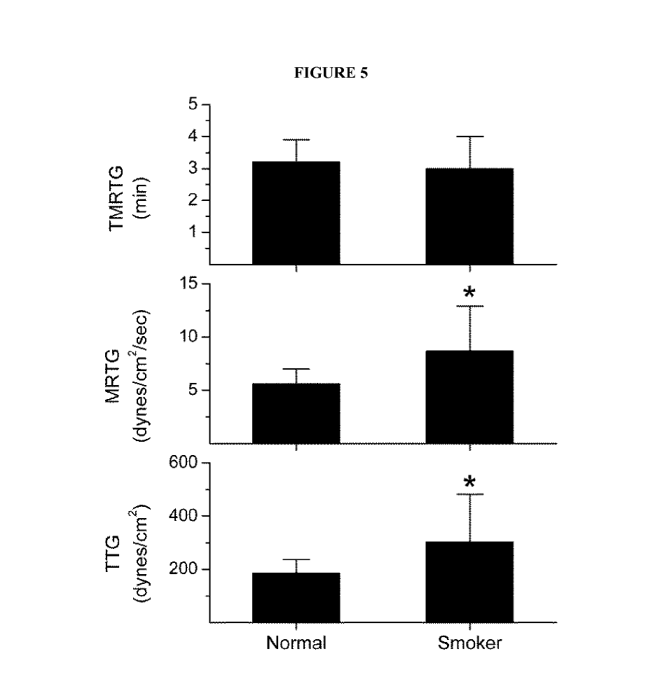

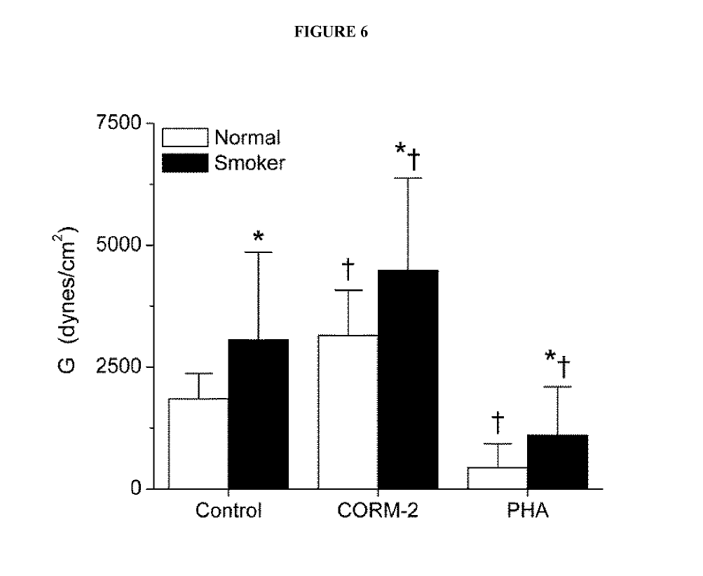

Smoking subjects, free of thrombotic or hemorrhagic disease, between the ages of 19 to 50 years of age were recruited (n=20). All comparative data are presented as mean±SD. The male:female ratio (M:F) was 14:6, and the smoking group's age was 32.8±8.7 years. This cohort smoked 1.0±0.4 packs of cigarettes per day. After written consent was obtained, the smoking subjects verified that they had smoked two cigarettes within 90 minutes prior to presentation as they would ordinarily do during the day. Noninvasive pulse oximetry was performed, with COHb found to be 5.0±2.7% for the smoking cohort. Whole blood was obtained from the smokers via venipuncture of an antecubital or hand vein and was anticoagulated with sodium citrate (9 parts blood to 1 part citrate). The blood was immediately centrifuged at 3000×g for 15 minutes at room temperature, with plasma decanted, aliquoted and stored at −80° C. prior to experimentation. Frozen, citrated plasma collected from 20 nonsmoking, age-matched normal subjects (30.7±8.7 years, M:F of 10:10) was obtained from a commercial source. Smoker and normal subject plasma was thawed and subjected to thrombelastographic analyses as previously described for 15 min at 37° C. (Nielsen et al., 2011, Blood Coagul Fibrinolysis, 22: 657-661). As seen in FIG. 5, while the onset of clot formation (time to maximum rate of thrombus formation, TMRTG) did not significantly change with smoking, the velocity of thrombus formation (maximum rate of thrombus generation, MRTG) and clot strength (total thrombus generation, TTG) was significantly (*P<0.01) increased in smoking subjects compared to normal subjects. This pattern of enhanced clot development without decreased time to onset of coagulation is typical of CO exposure (Nielsen et al., 2009, Blood Coagul Fibrinolysis, 20: 377-380; Nielsen et al., 2010, Blood Coagul Fibrinolysis, 21: 349-353; Nielsen et al., 2011, Blood Coagul Fibrinolysis, 22: 657-661). Plasma from the two groups' were subsequently exposed to no additive (vehicle alone), 100 μM CORM-2 or 30 mM PHA, with final elastic modulus (static strength, G) determined as displayed in FIG. 6. Smoking resulted in significantly enhanced G under all circumstances (*P<0.01). Further, within group, CORM-2 exposure significantly increased G and PHA exposure significantly decreased G (†P<0.012 vs. Control) as previously seen with the COHF assay (Nielsen et al., 2011, Blood Coagul Fibrinolysis, 22: 657-661). Responsiveness to CORM-2 indicated that there were heme groups not bound with CO present on fibrinogen. Decreased clot strength in response to PHA demonstrated the effects of a methemefibrinogen state; and specifically that there is a CO-independent enhancement of clot strength secondary to smoking that may be composed of increased fibrinogen/FXIII concentration as previously described (Lind et al., 2004, Arterioscler Thromb Vasc Biol, 24: 577-582; Ariens et al., 1999, Arterioscler Thromb Vasc Biol, 19: 2012-2016; van Wersch et al., 1997, Int J Clin Lab Res, 1997, 27: 68-71). Using a modification of the previously described assay (Nielsen et al., 2011, Blood Coagul Fibrinolysis, 22: 657-661), hypercoagulability was defined as a G value 95% confidence interval value of the normal cohort (2720 dynes/cm²). For COHF likely to be present, G values had to increase≦the average percent seen in normal subjects (≦78%) in the presence of CORM-2, and G values had to decrease≦the average normal percent (≦74%) in the presence of PHA. Using these definitions, 10 smokers were hypercoagulable; 9 smokers had kinetically detectable COHF; and 5 were both hypercoagulable and COHF positive (25% of the cohort). Of interest, 3 smokers had neither hypercoagulability nor detectable COHF (15% of the cohort), with the remaining 12 smokers either hypercoagulable only or COHF positive only (60% of the population). Bearing in mind that the present cohort was free of clinical thrombotic disease, the variation in coagulation response to smoking and CO exposure reflects historical experience—despite tobacco usage being a major risk factor for major cardiovascular events (Agarwal, 2009, Angiology, 60: 335-345; Bhatt et al., 2006, JAMA, 295: 180-189; Shah et al., 2010, Expert Rev Cardiovasc Ther., 8: 917-932; Oliveria et al., 2007, Preventative Medicine, 44: 311-316; Herman et al., 2011, Am J Public Health, 101: 491-496), not all smokers suffer thrombotic disease. Therefore, determination of vulnerability to CO-mediated hypercoagulability may identify the subpopulation of smokers at highest risk for thrombotic events, as well as identify the subpopulation of smokers perhaps best described as the “invulnerables”. Indeed, if the mechanism(s) by which these smoking but invulnerable individuals resist progression of hypercoagulability and/or detectable COHF formation can be identified, perhaps these insights may be used to design rational molecular therapies to attenuate the effects of smoking/CO exposure for the population at large.

### Example 4

### Detection of Carboxyhemefibrinogen and Methemefibrinogen in a Patient with Thrombosis of a Heartmate II Ventricular Assist Device

Endogenous carbon monoxide (CO) generation can be induced during hemolytic states that result in an increase in circulating free heme released from erythrocyte-derived hemoglobin (Coburn et al., 1964, J Clin Invest, 43: 1098-1103; Landaw et al, 1970, J Clin Invest, 49: 914-925). Ventricular assist devices (VADs) are known to damage red blood cells to a finite extent under the best of circumstances (Stepanenko et al., 2011, ASAIO J, 57: 382-387) and can at worst induce gross hemolysis (Sibbald et al., 2012, Catherization and Cardiovascular Inverventions, DOI 10.1002/ccd). Of interest, VAD therapy is associated with significant acquired hypercoagulability, requiring important systemic anticoagulation to prevent either thromboembolism from the device or frank thrombosis of the VAD (Nielsen et al., 2008, ASAIO J, 54: 351-358). Further, it has been recently discovered that CO released from a carbon monoxide releasing molecule (tricarbonyldichlororuthenium (II) dimer, CORM-2) significantly enhances the speed of clot formation and strength by binding to a heme group attached to fibrinogen (Nielsen et al., 2009, Blood Coagul Fibrinolysis, 20: 377-380; Nielsen et al., 2011, Blood Coagul Fibrinolysis, 22: 657-661). The primary molecule enhanced by CO is fibrinogen, designated carboxyhemefibrinogen (COHF) (Nielsen et al., 2011, Blood Coagul Fibrinolysis, 22: 657-661). Presented herein is a case of a patient with a Heartmate II (Thoratec Corporation, Pleasanton, Calif., USA) VAD that presented with partial thrombosis of the VAD, frank hemolysis and increased circulating carboxyhemoglobin (COHb) concentrations.

Case Report

The patient was a 71 year old male that had received a Heartmate II as destination therapy for ischemic cardiomyopathy one month prior to presentation. He presented acutely with symptoms of fatigue and return of pulsatile flow. Examination of the VAD demonstrated a marked increase in utilized current with minimal flow by transthoracic echocardiography. He had been anticoagulated with warfarin, aspirin, dipyridamole, and pentoxifylline. H is initial laboratory values were remarkable for a lactate dehydrogenase activity of 2717 U/ml, an international normalized ratio of 2.5, a fibrinogen concentration of 293 mg/dl, an arterial COHb concentration of 3.1-3.8% and a methemoglobin (MetHb) concentration of 0.9-1.7%. The patient was administered antithrombin and heparin as bridging anticoagulation, with an activated partial thromboplastin time of 60.9 seconds achieved. The patient was emergently prepared for surgical replacement of the thrombosed VAD.

Shortly after induction of anesthesia and invasive monitoring placement, whole blood was collected from the patient's radial arterial catheter and immediately anticoagulated with sodium citrate (9 parts blood:1 part 0.105M sodium citrate). The blood was immediately centrifuged at 3000×g at room temperature, with plasma aliquoted and stored at −80° C. prior to thrombelastographic analyses subsequently described. Operation proceeded, with VAD replacement followed by discharge from the hospital three weeks later.

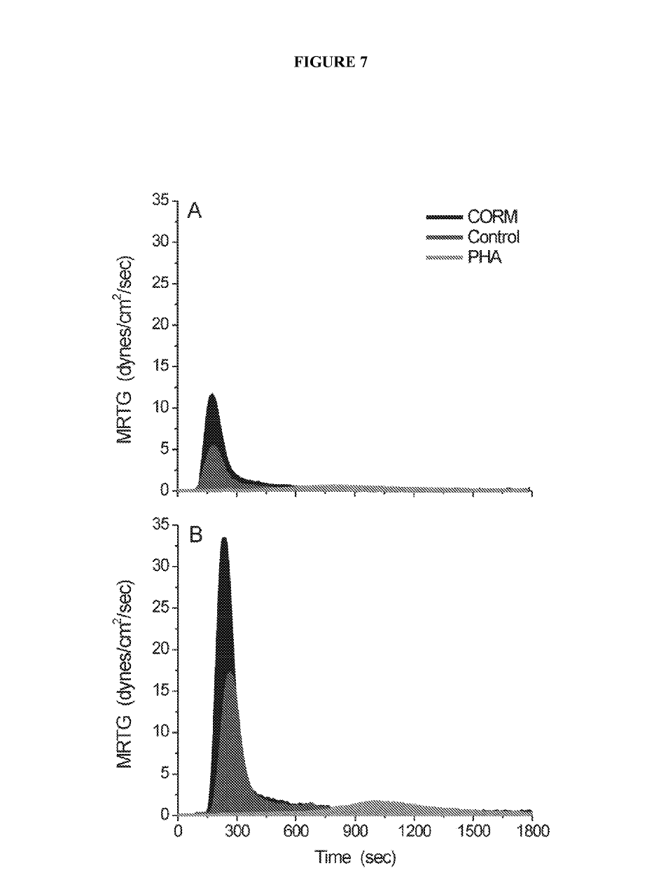

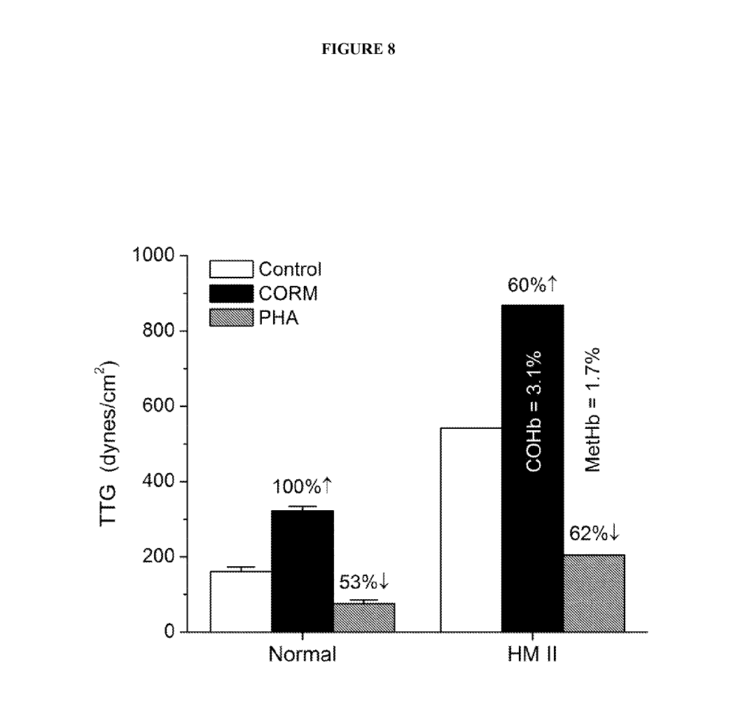

Patient plasma was subjected to thrombelastographic analysis for 30 min at 37° C. as previously described (Nielsen et al., 2009, Blood Coagul Fibrinolysis, 20: 377-380; Nielsen et al., 2011, Blood Coagul Fibrinolysis, 22: 657-661). Also, for qualitative/semi-quantitative comparison, four replicate samples of citrated, pooled normal plasma (George King Bio-Medical, Overland Park, Kans., USA) were subjected to identical thrombelastographic analyses. In brief, the final volume for all subsequently described plasma sample mixtures was 359.6 Sample composition consisted of 326 μl of plasma; 10 μl of tissue factor reagent (0.1% final concentration in dH₂O; Diagnostica Stago S.A.S., Asnieres sur Seine, France), 3.6 μl of dH₂O or dH₂O containing the organic reductant phenylhydroxylamine (PHA, 30 mM final concentration, Sigma-Aldrich, Saint Louis, Mo., USA), or 3.6 μl of CORM-2 (100 μM final concentration; Sigma-Aldrich) and 20 μl of 200 mM CaCl₂. The addition of CORM-2 determines if there are heme groups unbound by CO in plasma by increasing coagulation kinetics (Nielsen et al., 2011, Blood Coagul Fibrinolysis, 22: 657-661). PHA at this concentration rapidly converts heme-attached Fe⁺² to Fe⁺³, the met-state, displacing CO and decreasing coagulation (Nielsen et al., 2011, Blood Coagul Fibrinolysis, 22: 657-661). Standard heparinase-containing cups were used for all samples to eliminate the effects of systemic heparin administration received by the patient. A computer-controlled Thrombelastograph® hemostasis system (Model 5000, Haemoscope Corp., Niles, Ill., USA) was used, with addition of CaCl₂ as the last step to initiate clotting. Thrombelastographic variables previously described (Nielsen et al., 2009, Blood Coagul Fibrinolysis, 20: 377-380; Nielsen et al., 2011, Blood Coagul Fibrinolysis, 22: 657-661) were determined, and the results are depicted in FIG. 7 and FIG. 8.

As seen in FIG. 7, compared to a typical normal sample (panel A), the VAD patient's plasma sample had an approximately three-fold greater speed of growth and strength (panel B) in plasma without additions and in plasma exposed to either CORM-2 or PHA. Of interest, as seen in FIG. 8, while normal plasma had a typical increase in strength and in response to CORM-2, the VAD patient sample had a markedly decreased response, indicative of the presence of COHF (Nielsen et al., 2011, Blood Coagul Fibrinolysis, 22: 657-661), in the presence of increased COHb. However, compared to normal plasma, the VAD patient had a greater decrease in clot strength after exposure to PHA, likely secondary to the presence of methemefibrinogen (MetHF) from exposure to endogenous NO, evidenced by increased MetHb.

Hypercoagulability in a VAD Patient

The presented case is the first report of hypercoagulability/thrombophilia in a VAD patient with increased CO production secondary to hemolysis with concurrent increased COHb and increased COHF. However, data presented herein shows a mixed coagulopathy, with the presence of MetHF detected with concurrent increased MetHb. Not surprisingly, the summation of hemostatic effect of simultaneous exposure to CO and NO was hypercoagulability, as a far greater concentration (100-fold) of metheme producing agent is required to attenuate the prohemostatic effects of CO in plasma (Nielsen et al., 2011, Blood Coagul Fibrinolysis, 22: 657-661). In sum, the patient suffered device thrombosis with important plasmatic hypercoagulability and increased COHF concentrations despite conventional plasmatic and antiplatelet anticoagulation therapies.

While encouraging, the data is preliminary and cannot implicate increased COHF concentrations as the primary cause of the patient's device thrombosis. It is possible that free heme release caused by the device upregulated the hemeoxygenase-1 system (Coburn et al., 1964, J Clin Invest, 43: 1098-1103; Landaw et al, 1970, J Clin Invest, 49: 914-925), which in turn increased circulating CO, increased COHF formation, and subsequently accelerated thrombus formation on the Heartmate II with consequent thrombosis resulting. However, it is also possible that progressive thrombus formation on the device occurred secondary to some other thrombin-dependent or platelet-dependent process unrelated to CO formation, with COHF formation occurring later. Presented herein is a case of thrombosis of a Heartmate II VAD with concurrent plasmatic hypercoagulability and increased circulating COHF and COHb concentrations secondary to upregulation of the hemoxygenase-1 system via device-related hemolysis.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of diagnosing hypercoagulability associated with carbon monoxide exposure in a subject, the method comprising:
a. obtaining a plasma sample from the subject, and
b. dividing the plasma sample into at least two portions, and
c. determining the clot strength of a first portion of the plasma sample, and
d. exposing a second portion of the plasma sample to an effective amount of an organic reductant, and
e. determining the clot strength of the second portion of the plasma sample after exposure to the effective amount of the organic reductant, and
f. comparing the clot strength of the first portion of the plasma sample with the clot strength of the second portion of the plasma sample, wherein when the clot strength of the second portion of the plasma sample is decreased compared with the clot strength of the first portion of the plasma sample, the subject is diagnosed with hypercoagulability associated with carbon monoxide exposure.

2. The method of claim 1, wherein the hypercoagulability is associated with carboxyhemefibrinogen formation.

3. The method of claim 1, wherein the clot strength of the first portion of the plasma sample is greater than an institutionally generated normal 95% confidence interval.

4. The method of claim 1, wherein the clot strength of the second portion of the plasma sample is decreased by at least 35% as compared with the clot strength of the first portion of the plasma sample.

5. The method of claim 1, wherein the strength of the clot is determined by measuring the elastic modulus (G).

6. The method of claim 1, wherein the clot strength is determined by using a thromboelastograph or a thromboelastometer.

7. The method of claim 1, wherein the subject is a mammal.

8. The method of claim 1, wherein the organic reductant is at least one selected from the group consisting of phenyhydroxylamine, phenylhydrazine, sym-diphenylhydrazine, 1,4-cyclohexane, hydroquinone, benzhydrol, methylhydrazine, 2-methyl-1,3-cyclopentanedione, acetylacetone, isopropyl alcohol, benzaldehyde, and malononitrile.

9. The method of claim 1, wherein the effective amount of the organic reductant is sufficient to produce a concentration of 30 mM.

10. The method of claim 1, wherein the effective amount of the organic reductant is sufficient to produce a concentration of 5-500 mM, 10-400 mM, 15-300 mM, 20-200 mM, or 25-100 mM.

11. The method of claim 1, wherein when the addition of a carbon monoxide-producing molecule to a third portion of the plasma sample does not increase the clot strength of the third portion of the plasma sample by at least 35% as compared with the clot strength of the first portion of the plasma sample, the diagnosis of hypercoagulability associated with carbon monoxide exposure is confirmed.

12. The method of claim 11, wherein the carbon monoxide producing molecule is at least one selected from the group consisting of: tricarbonyldichlororuthenium (II) dimer ([RuCl2(CO)3]2; carbon monoxide releasing molecule-2; CORM-2), iron pentacarbonyl (Fe(CO)5), dimanganese decacarbonyl (Mn2(CO)10; CORM-1), tricarbonylchloro(glycinato)ruthenium (II) (Ru(CO)3Cl(glycinate); CORM-3), and sodium boranocarbonate (Na2[H3BCO2]; CORM-A1).

13. The method of claim 11, wherein the effective amount of the carbon monoxide producing molecule is sufficient to produce a concentration of 100 μM.

14. The method of claim 11, wherein the effective amount of the carbon monoxide producing molecule is sufficient to produce a concentration of 5-500 μM, 10-400 μM, 15-300 μM, 20-200 μM, or 25-100 μM.

15. A method of diagnosing hypocoagulability associated with nitric oxide exposure in a subject, the method comprising:
a. obtaining a plasma sample from the subject, and
b. dividing the plasma sample into at least two portions, and
c. determining the clot strength of a first portion of the plasma sample, and
d. exposing a second portion of the plasma sample to an effective amount of a carbon monoxide-producing molecule, and
e. determining the clot strength of the second portion of the plasma sample after exposure to the effective amount of the carbon monoxide-producing molecule, and
f. comparing the clot strength of the first portion of the plasma sample with the clot strength of the second portion of the plasma sample, wherein when the clot strength of the second portion of the plasma sample is increased compared with the clot strength of the first portion of the plasma sample, the subject is diagnosed with hypocoagulability associated with nitric oxide exposure.

16. The method of claim 15, wherein the hypocoagulability is associated with methemefibrinogen formation.

17. The method of claim 15, wherein the clot strength of the first portion of the plasma sample is less than an institutionally generated normal 95% confidence interval.

18. The method of claim 15, wherein the clot strength of the second portion of the plasma sample is increased by at least 160% as compared with the clot strength of the first portion of the plasma sample.

19. The method of claim 15, wherein the strength of the clot is determined by measuring the elastic modulus (G).

20. The method of claim 15, wherein the clot strength is determined by using a thromboelastograph or a thromboelastometer.

21. The method of claim 15, wherein the subject is a mammal.

22. The method of claim 15, wherein the carbon monoxide producing molecule is at least one selected from the group consisting of: tricarbonyldichlororuthenium (II) dimer ([RuCl2(CO)3]2; carbon monoxide releasing molecule-2; CORM-2), iron pentacarbonyl (Fe(CO)5), dimanganese decacarbonyl (Mn2(CO)10; CORM-1), tricarbonylchloro(glycinato)ruthenium (II) (Ru(CO)3Cl(glycinate); CORM-3), and sodium boranocarbonate (Na2[H3BCO2]; CORM-A1).

23. The method of claim 15, wherein the effective amount of the carbon monoxide producing molecule is sufficient to produce a concentration of 100 μM.

24. The method of claim 15, wherein the effective amount of the carbon monoxide producing molecule is sufficient to produce a concentration of 5-500 μM, 10-400 μM, 15-300 μM, 20-200 μM, or 25-100 μM.

25. The method of claim 15, wherein when the addition of an organic reductant to a third portion of the plasma sample does not decrease the clot strength of the third portion of the plasma sample by at least 35% as compared with the clot strength of the first portion of the plasma sample, the diagnosis of hypocoagulability associated with nitric oxide exposure is confirmed.

26. The method of claim 25, wherein the organic reductant is at least one selected from the group consisting of phenyhydroxylamine, phenylhydrazine, sym-diphenylhydrazine, 1,4-cyclohexane, hydroquinone, benzhydrol, methylhydrazine, 2-methyl-1,3-cyclopentanedione, acetylacetone, isopropyl alcohol, benzaldehyde, and malononitrile.

27. The method of claim 25, wherein the effective amount of the organic reductant is sufficient to produce a concentration of 30 mM.

28. The method of claim 25, wherein the effective amount of the organic reductant is sufficient to produce a concentration of 5-500 mM, 10-400 mM, 15-300 mM, 20-200 mM, or 25-100 mM.
